(19) United States
(12) Patent Application Publication
(10) Pub. No.: US 2026/0252877 A1
(43) Pub. Date: Aug. 27, 2026

(54) SYSTEM

(71) Applicant: SoftBank Group Corp., Tokyo (JP)

(72) Inventor: Kazuhiro KAMAKURA, Tokyo (JP)

(73) Assignee: SoftBank Group Corp., Tokyo (JP)

(21) Appl. No.: 19/533,365

(22) Filed: Feb. 9, 2026

(30) Foreign Application Priority Data
Feb. 21, 2025 (JP) 2025-027011

(51) Int. Cl. G06N 3/08; G06N 3/0455; G06N 3/0475

(52) U.S. Cl. CPC G06N 3/08; G06N 3/0455; G06N 3/0475

(57) ABSTRACT

The system according to the embodiment comprises a collection unit, a learning unit, a detection unit, and a guidance unit. The collection unit collects health status or attendance information of employees. The learning unit learns information collected by the collection unit. The detection unit detects changes in health status based on information learned by the learning unit. The guidance unit provides guidance on treatment or consultation destinations based on changes in health status detected by the detection unit.

### CROSS-REFERENCE TO RELATED APPLICATION

The present application claims priority to and incorporates by reference the entire contents of Japanese Patent Application No. 2025-027011 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to efficiently manage employees' health status and attendance information and to provide guidance on appropriate treatment or consultation destinations.

### SUMMARY OF THE INVENTION

The system according to the embodiment comprises a collection unit, a learning unit, a detection unit, and a guidance unit. The collection unit collects health status or attendance information of employees. The learning unit learns information collected by the collection unit. The detection unit detects changes in health status based on information learned by the learning unit. The guidance unit provides guidance on treatment or consultation destinations based on changes in health status detected by the detection unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

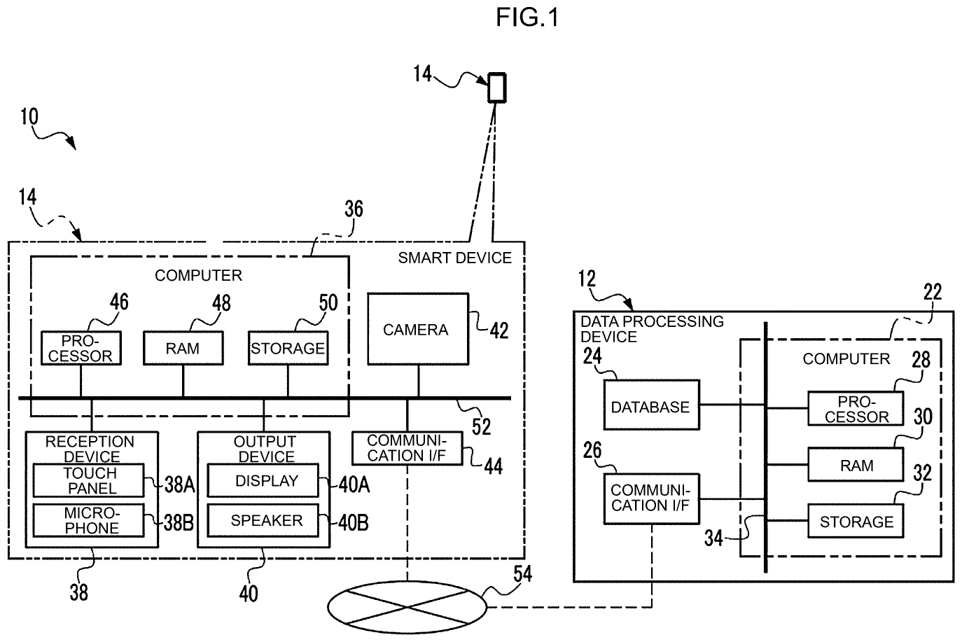

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

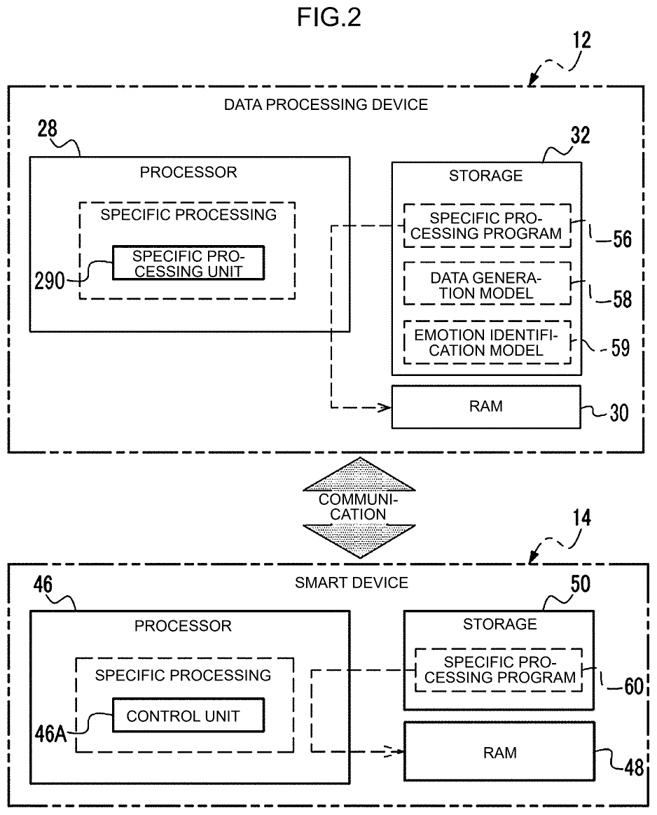

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is a system that uses AI to learn information on employees' health status, attendance, and work details, and provides guidance on appropriate treatment or consultation destinations. This system enables AI to learn daily information on health status, attendance, and work details. Next, the AI asks light questions about employees' health status via chat tools such as communication tools. For example, it may ask, “Your sleep duration decreased yesterday, was there anything wrong?” The AI considers changes in health status and the content of message replies, and provides guidance on appropriate treatment or consultation destinations to the employee or their supervisor. As a result, the system can maintain employees in a healthy working state and also visualize teams that are experiencing burnout. For example, the AI learns daily information on health status, attendance, and work details. At this time, detailed data such as employees' sleep duration, physical condition, working hours, and work details are collected and learned by the AI. For example, the AI analyzes health status and attendance information entered daily by employees and learns patterns. Next, the AI asks light questions about employees' health status via communication tools. For example, it sends questions such as “Your sleep duration decreased yesterday, was there anything wrong?” via chat tools. This enables the system to grasp changes in employees' health status. The AI considers changes in health status and the content of message replies, and provides guidance on appropriate treatment or consultation destinations to the employee or their supervisor. For example, if an employee replies, “Recently, I can't sleep due to work stress,” the AI uses that information to guide the employee to a stress management expert or counseling service. In addition, the supervisor is notified of the employee's health status and prompted to take appropriate action. As a result, the system can maintain employees in a healthy working state. For example, by receiving appropriate treatment, employees can reduce physical discomfort and stress, leading to improved work efficiency. Furthermore, the system can visualize teams that are experiencing burnout. For example, if multiple employees in the same team have health problems, the AI uses that information to visualize the overall team situation and propose appropriate countermeasures. Thus, the AI system can maintain employees in a healthy working state by grasping their health status and providing guidance on appropriate treatment or consultation destinations. Specifically, the system is composed of multiple modules, and the collection unit collects multidimensional data for each employee on sleep duration (e.g., 6.5 hours, 5.0 hours, 8.0 hours), physical condition (e.g., fatigue score 70/100, high stress level, body temperature 36.8° C.), working hours (e.g., 9:00-18:00, 2 hours overtime), and work details (e.g., desk work, field work, number of meetings attended) on a daily basis. The system manages these data as time-series tensors (e.g., a three-dimensional array of number of employees ×number of days ×number of features), and the preprocessing unit performs preprocessing such as missing value imputation, outlier removal, and normalization (e.g., z-score standardization). The learning unit uses, for example, recurrent neural networks (RNN) or transformer models to learn time-series patterns of health status, attendance, and work details for each employee. As an input example, a vector sequence of sleep duration, physical condition, working hours, and work details for one week (e.g., 7×10 dimensions) is provided, and as output, a “health status change score” or “abnormality probability distribution” (e.g., 0.85=high risk, 0.15=low risk) is generated. Furthermore, the learning unit analyzes employees' past reply histories and chat contents (e.g., text data such as “Recently, I can't sleep,” “I'm not feeling well”) using natural language processing models (e.g., BERT-based classifiers) to extract emotion labels (e.g., stress, fatigue, normal) and risk factors (e.g., excessive workload, family problems). The detection unit applies threshold judgment (e.g., health status change score>0.7 is abnormal) and rule-based branching (e.g., stress label+decreased sleep duration is a warning) to these output values to automatically extract abnormal states or warning targets. The guidance unit selects optimal treatment proposals (e.g., occupational physician interview, stress check, recommendation to take leave) or consultation destinations (e.g., in-house counselor, external consultation desk) from a database of pre-learned expert advice and counseling service lists based on the judgment results of the detection unit, and automatically notifies the employee or supervisor via chat tools. AI-generated questions are triggered by recent changes in employee data, and natural questions such as “Your sleep duration decreased yesterday, how is your physical condition?” are generated using template-based or generative models (e.g., GPT series) and sent via chat tool APIs. Input examples to the AI include time-series vectors of sleep duration (e.g., 7 days), physical condition scores, recent attendance changes, and past reply texts (e.g., “I worked a lot of overtime yesterday”), and output examples include health status change score 0.82, emotion label “stress,” and recommended treatment “counseling guidance.” These outputs are used for subsequent notification modules, dashboard displays, and report generation for supervisors. As a technical effect, this system, unlike conventional simple health management or attendance supervision by humans, can detect changes in health status and signs of team burnout early and with high accuracy by integrating high-dimensional data analysis, time-series pattern recognition, natural language understanding, and automatic application of anomaly detection algorithms using AI. This prevents employee health risks in advance and contributes to improved work efficiency and overall organizational productivity. In addition, the AI's automatic questioning, notification, and visualization functions greatly reduce the burden on employees and managers, and realize objective and highly reproducible health management that does not depend on manual work or subjective judgment. Specific application fields include project management in IT companies, health monitoring of field workers in manufacturing, stress management in call centers, and health maintenance support in remote work environments, and can be deployed in various industries and occupations.

The health management system according to the embodiment comprises a collection unit, a learning unit, a detection unit, and a guidance unit. The collection unit collects health status or attendance information of employees. Health status of employees includes, for example, body temperature, blood pressure, heart rate, but is not limited thereto. Attendance information includes, for example, clock-in time, clock-out time, break time, but is not limited thereto. The collection unit collects, for example, health status and attendance information entered daily by employees. The learning unit learns information collected by the collection unit. The learning unit analyzes the collected information using, for example, machine learning or deep learning, and learns patterns. The detection unit detects changes in health status based on information learned by the learning unit. The detection unit uses, for example, an algorithm to detect abnormal values by comparing with past data. The guidance unit proposes appropriate treatment or consultation destinations based on changes in health status detected by the detection unit. The guidance unit proposes appropriate treatment based on, for example, pre-learned expert advice or information on counseling services. Thus, the health management system according to the embodiment can maintain employees in a healthy working state by grasping their health status and providing guidance on appropriate treatment or consultation destinations. Specifically, the health management system collects multidimensional data for each employee on body temperature (e.g., 36.5° C., 37.2° C.), blood pressure (e.g., 120/80 mmHg, 135/90 mmHg), heart rate (e.g., 72 bpm, 85 bpm), clock-in time (e.g., 9:00), clock-out time (e.g., 18:00), break time (e.g., 12:00-13:00) on a daily basis. The system manages these data as a three-dimensional tensor of number of employees×number of days×number of features, and the preprocessing unit performs preprocessing such as missing value imputation (e.g., mean imputation), outlier removal (e.g., 3 σ method), and normalization (e.g., z-score standardization). The learning unit uses, for example, recurrent neural networks (RNN) or transformer models to learn time-series patterns of health status and attendance for each employee. Input examples to the AI include a vector sequence of body temperature, blood pressure, heart rate, clock-in/clock-out/break time for one week (e.g., 7×6 dimensions), and as output, a “health status change score” (e.g., 0.82) and abnormality probability distribution (e.g., 0.85=high risk, 0.15=low risk) are generated. The detection unit applies threshold judgment (e.g., health status change score>0.7 is abnormal) and rule-based branching (e.g., increased heart rate+late arrival is a warning) to these output values to automatically extract abnormal states or warning targets. The guidance unit selects optimal treatment proposals (e.g., occupational physician interview, stress check, recommendation to take leave) or consultation destinations (e.g., in-house counselor, external consultation desk) from a database of pre-learned expert advice and counseling service lists based on the judgment results of the detection unit, and automatically notifies the employee or supervisor via chat tools. AI-generated questions are triggered by recent changes in employee data, and natural questions such as “Your heart rate has been high recently, how is your physical condition?” are generated using template-based or generative models and sent via chat tool APIs. Input examples to the AI include time-series vectors of body temperature, blood pressure, heart rate, recent attendance changes, and past reply texts (e.g., “I worked a lot of overtime yesterday”), and output examples include health status change score 0.82 and recommended treatment “counseling guidance.” These outputs are used for subsequent notification modules, dashboard displays, and report generation for supervisors. As a technical effect, this system, unlike conventional simple health management or attendance supervision by humans, can detect changes in health status and risk signs early and with high accuracy by integrating high-dimensional data analysis, time-series pattern recognition, and automatic application of anomaly detection algorithms using AI. This prevents employee health risks in advance and contributes to improved work efficiency and overall organizational productivity. In addition, the AI's automatic questioning, notification, and visualization functions greatly reduce the burden on employees and managers, and realize objective and highly reproducible health management that does not depend on manual work or subjective judgment. Specific application fields include project management in IT companies, health monitoring of field workers in manufacturing, stress management in call centers, and health maintenance support in remote work environments, and can be deployed in various industries and occupations.

The collection unit can collect detailed data on employees' sleep duration, physical condition, working hours, and work details. The collection unit collects, for example, detailed data on employees' sleep duration, physical condition, working hours, and work details. Physical condition includes, for example, fatigue level, stress level, but is not limited thereto. Work details include, for example, desk work, field work, but are not limited thereto. By collecting detailed data on employees, it is possible to more accurately grasp their health status. Specifically, the collection unit collects multidimensional data for each employee on sleep duration (e.g., 6.5 hours, 5.0 hours, 8.0 hours), physical condition scores (e.g., fatigue level 70/100, high stress level, body temperature 36.8° C.), working hours (e.g., 9:00-18:00, 2 hours overtime), and work details (e.g., desk work, field work, number of meetings attended: 3) on a daily basis. The collection unit manages these data as a three-dimensional tensor of number of employees×number of days×number of features and stores them in a database. Furthermore, the collection unit can automatically acquire data from multiple data sources such as wearable devices, attendance management systems, and work report systems. Input examples to the AI include a vector sequence of sleep duration, physical condition, working hours, and work details for one week (e.g., 7×10 dimensions), which are preprocessed for missing value imputation and normalization before being passed to the learning unit. The collection unit integrates not only subjective physical condition scores and stress levels entered by employees, but also objective biometric sensor data and automatically acquired data from business systems, thereby improving the reliability and coverage of the data. As a technical effect, the collection unit, unlike conventional simple self-reporting or paper-based records, can automatically collect and integrate high-dimensional and time-series detailed data from diverse data sources, enabling high-precision and real-time grasp of changes in health status and risk factors. As a result, the accuracy of AI analysis and anomaly detection is greatly improved, enabling early detection of employee health risks and appropriate countermeasure proposals. Specific application fields include health monitoring of field workers, stress management of office workers, and health maintenance support in remote work environments.

The learning unit can analyze the collected information and learn patterns. The learning unit analyzes the collected information and learns patterns, for example. Patterns include, for example, temporal patterns, behavioral patterns, but are not limited thereto. By analyzing the collected information and learning patterns, changes in health status can be detected more accurately. Specifically, the learning unit receives time-series tensor data (e.g., number of employees×number of days×number of features) from the collection unit as input, and uses deep learning architectures such as recurrent neural networks (RNN), long short-term memory networks (LSTM), or transformer models to learn time-series patterns of health status, attendance, and work details for each employee. Input examples to the AI include a vector sequence of sleep duration, physical condition, working hours, and work details for one week (e.g., 7×10 dimensions), and a one-month transition of physical condition scores (e.g., 30×3 dimensions). The learning unit applies feature extraction layers (e.g., convolutional layers, self-attention mechanisms) to these data to extract latent patterns indicating changes in health status or abnormal signs. As output, it generates a “health status change score” (e.g., 0.82), “abnormality probability distribution” (e.g., 0.85=high risk, 0.15=low risk), and “behavior pattern labels” (e.g., night-type tendency, increasing overtime tendency). The learning unit optimizes model parameters using loss functions (e.g., cross-entropy, mean squared error) and improves pattern recognition accuracy through supervised or self-supervised learning. Furthermore, the learning unit combines techniques such as data augmentation (e.g., time-series shift, noise addition) and weighted learning (e.g., emphasis on latest data) to enhance model generalization performance. As a technical effect, the learning unit, unlike conventional simple statistical analysis or rule-based processing, can detect subtle changes in health status and complex behavioral patterns with high accuracy by automatic feature extraction and pattern learning of high-dimensional and time-series data using AI. As a result, the accuracy of anomaly detection and risk prediction is greatly improved, contributing to employee health maintenance and work efficiency. Specific application fields include health risk prediction, stress factor analysis, and optimization of work patterns.

The detection unit can use an algorithm to detect abnormal values by comparing with past data. The detection unit uses, for example, an algorithm to detect abnormal values by comparing with past data. Abnormal values include, for example, degree of deviation from the normal range, abnormal value thresholds, but are not limited thereto. By detecting abnormal values by comparing with past data, changes in health status can be discovered early. Specifically, the detection unit receives health status change scores and abnormality probability distributions output by the learning unit as input, and applies anomaly detection algorithms (e.g., time-series anomaly detection, autoencoder-based anomaly scoring, rule-based threshold judgment). Input examples to the AI include a series of health status change scores for the past week (e.g., 0.65, 0.68, 0.82, 0.90, 0.75, 0.80, 0.85), and a one-month transition of physical condition scores (e.g., 30 days of continuous values). The detection unit compares these series data with past normal ranges (e.g., individual mean±2 σ), and judges as abnormal when the degree of deviation exceeds a threshold (e.g., 0.7). Furthermore, the detection unit can apply composite rules such as simultaneous changes in multiple features (e.g., decreased sleep duration+increased heart rate) or consecutive occurrence of abnormal patterns (e.g., abnormal score exceeding threshold for 3 consecutive days). As output, it generates an “anomaly detection flag” (e.g., 1=abnormal, 0=normal), “abnormality score” (e.g., 0.85), and “abnormality factor label” (e.g., increased stress, tendency to overwork). These outputs are used by subsequent guidance units and notification modules, and utilized for alerts and report generation for employees and managers. As a technical effect, the detection unit, unlike conventional simple threshold judgment or manual monitoring, can discover changes in health status and risk signs early and with high accuracy by time-series anomaly detection and composite feature analysis using AI. As a result, employee health risks can be prevented in advance and rapid response is possible, contributing to improved productivity of the entire organization. Specific application fields include early detection of health abnormalities, automatic detection of excessive stress, and automatic alerts for attendance abnormalities.

The guidance unit can propose appropriate treatment based on pre-learned expert advice or information on counseling services. The guidance unit proposes appropriate treatment based on, for example, pre-learned expert advice or information on counseling services. Expert advice includes, for example, medical diagnosis, counselor advice, but is not limited thereto. Counseling services include, for example, online counseling, face-to-face counseling, but are not limited thereto. By proposing appropriate treatment based on expert advice or information on counseling services, employees' health status can be improved. Specifically, the guidance unit receives anomaly detection flags and abnormality factor labels output by the detection unit as input, and automatically selects optimal treatment proposals and consultation destinations from a database of pre-learned expert advice (e.g., recommendation for occupational physician interview, stress check, recommendation to take leave) and counseling service lists (e.g., in-house counselor, external consultation desk, online consultation service). Input examples to the AI include abnormality factor label “increased stress,” health status change score 0.85, and past consultation history (e.g., date of last counseling session). The guidance unit judges priority and urgency based on this information and generates optimal guidance content (e.g., “Please consult a stress management expert,” “We recommend an occupational physician interview”). As output, it generates “recommended treatment proposals” (e.g., counseling guidance, recommendation to take leave), “guidance destination list” (e.g., in-house counselor, external consultation desk), and “notification messages” (e.g., “Your stress has been increasing recently. Let's consult an expert.”). These outputs are automatically notified to the employee or supervisor via chat tool APIs or dashboards. As a technical effect, the guidance unit, unlike conventional manual advice or manual response, realizes rapid and objective treatment guidance by automatic matching of AI anomaly detection results and expert knowledge. As a result, it contributes to improvement of employees' health status, risk reduction, and work efficiency. Specific application fields include automatic guidance for health consultation, stress management support, and automatic recommendation for occupational physician interviews.

The collection unit can estimate the emotions of employees and determine the frequency of data collection based on the estimated emotions of the employees. The collection unit estimates the emotions of employees and adjusts the frequency of data collection based on the estimated emotions, for example. Emotions include, for example, facial expression analysis, voice analysis, but are not limited thereto. Frequency of data collection includes, for example, setting collection intervals according to changes in emotions, but is not limited thereto. For example, if an employee is feeling stressed, the frequency of data collection is reduced to lessen the burden. If the employee is relaxed, the frequency of data collection can be increased to collect more detailed information. If the employee is tired, the frequency of data collection can be adjusted to collect data at appropriate times. By adjusting the frequency of data collection based on employees' emotions, their burden can be reduced. Specifically, the collection unit uses facial image data (e.g., 128×128 pixel RGB tensor), voice waveform data (e.g., 1 second of 16 kHz sampled PCM array), and transcribed speech content (e.g., natural language text such as “I'm tired recently”) as input for emotion estimation. The collection unit combines a facial recognition model using convolutional neural networks (CNN), a voice emotion classification model using spectrogram conversion and recurrent neural networks (RNN), and a natural language processing model (e.g., BERT-based emotion classifier) to estimate “emotion labels” (e.g., stress, relaxation, fatigue) and “emotion scores” (e.g., stress level 0.78, relaxation level 0.12) from each data type. Input examples to the AI include 10 facial images, 5 voice clips, and 3 speech texts per day, and output examples include emotion label “stress” and emotion score 0.78. The collection unit dynamically adjusts the collection interval, such as reducing the frequency from once per day to once every three days when the estimated emotion score is high (e.g., stress level>0.7). Conversely, when the relaxation level is high, more detailed data collection is performed twice per day, optimizing the frequency according to the emotional state. These frequency controls are personalized by linking with individual employee history databases and are coordinated with past emotion transitions and health status changes. As a technical effect, the collection unit, unlike conventional fixed-interval data collection, minimizes employees' psychological and physical burden while obtaining high-precision health data at necessary times by combining AI-based multimodal emotion estimation and dynamic frequency control algorithms. As a result, the quality and coverage of data are improved, and the accuracy of subsequent health status analysis and anomaly detection is greatly enhanced. Specific application fields include call center operations where stress management is important, safety monitoring of field workers, and health maintenance support in remote work environments, where both psychological burden and data quality must be balanced across various industries and occupations.

The collection unit can analyze employees' past health status or attendance information and select an appropriate data collection method. The collection unit analyzes employees' past health status or attendance information and selects the optimal data collection method, for example. Past health status includes, for example, past diagnosis results, health checkup data, but is not limited thereto. Attendance information includes, for example, clock-in time, clock-out time, break time, but is not limited thereto. For example, by analyzing employees' past health status, if health status is deteriorating, detailed data is collected. By analyzing attendance information, if working hours are long, data can be collected during break times. By analyzing work details, if workload is high, data can be collected after work. By analyzing employees' past health status or attendance information, the optimal data collection method can be selected. Specifically, the collection unit acquires each employee's past one year of health checkup data (e.g., time-series vectors of body temperature, blood pressure, heart rate, blood test values), attendance history (e.g., time-series tensors of clock-in/clock-out times, overtime hours, break acquisition status), and work report data (e.g., work details, workload scores) from the database. The collection unit applies anomaly detection algorithms (e.g., autoencoder-based anomaly scoring, time-series clustering) and pattern mining techniques (e.g., frequent pattern extraction, trend analysis) to these multidimensional time-series data to automatically extract health deterioration trends (e.g., continuous decrease in physical condition scores, increasing overtime hours) and attendance abnormalities (e.g., frequent lateness or early leave). Input examples to the AI include a series of physical condition scores for the past 30 days (e.g., 30×3 dimensions), attendance history (e.g., 30×4 dimensions), and workload scores (e.g., 30 days of continuous values), and output examples include “health deterioration flag 1,” “recommendation for detailed data collection,” and “collection timing: break time.” Based on these outputs, the collection unit dynamically optimizes the collection method, such as additionally collecting biometric sensor data from wearable devices or detailed subjective questionnaires for employees with detected health deterioration, while performing only standard data collection for employees with stable health status. Furthermore, when workload is high, the collection unit automatically selects times with less employee burden, such as after work or during breaks, and sends data collection requests. As a technical effect, the collection unit, unlike conventional uniform and fixed data collection, realizes optimal data collection according to each employee's health risk and work situation by combining AI-based past data analysis and dynamic collection method selection algorithms. As a result, the coverage and reliability of data are improved, and changes in health status and risk signs can be grasped with high accuracy while minimizing employee burden. Specific application fields include the IT industry where long working hours are a problem, health monitoring of field workers, and attendance anomaly detection for shift workers, and can be deployed in various industries and occupations.

The collection unit can perform filtering during data collection based on employees' current projects or workload. The collection unit performs filtering during data collection based on employees' current projects or workload, for example. Projects include, for example, development projects, marketing projects, but are not limited thereto. Workload includes, for example, amount of work, stress level, but is not limited thereto. For example, if an employee is working on an important project, data collection is withheld. If the employee's workload is high, data collection can be minimized. If the employee is engaged in light work, detailed data can be collected. By filtering data collection based on employees' current projects or workload, it is possible to avoid interfering with work. Specifically, the collection unit acquires project assignment data for each employee (e.g., project ID, role, progress status), workload scores (e.g., work amount index, stress level score), and recent work reports (e.g., work details, number of meetings attended) in real time. The collection unit applies project importance judgment algorithms (e.g., project progress rate, days until deadline, number of remaining tasks) and workload estimation models (e.g., stress score estimation, time-series change in work amount) to these data to quantitatively evaluate employees' work status. Input examples to the AI include current project ID “P123,” progress rate 80%, stress level 0.85, work amount index 120, and output examples include “recommendation to withhold data collection,” “collection frequency: once a week,” and “recommendation for detailed collection.” When important projects or high workload states are detected, the collection unit reduces the frequency of data collection and prioritizes employees' concentration on work, while actively collecting detailed data on health status and work details when workload is low. These filtering processes are dynamically optimized in conjunction with each employee's work history and project characteristics. As a technical effect, the collection unit, unlike conventional uniform data collection, can efficiently obtain necessary health and attendance data while minimizing the impact on work by combining AI-based workload estimation and project importance judgment. As a result, changes in health status and risk signs can be grasped with high accuracy without impairing employees' work efficiency or productivity. Specific application fields include development sites with strict deadlines, call centers during busy periods, and management work handling multiple projects in parallel, where both workload and health management are required.

The collection unit can estimate the emotions of employees and determine the priority of data to be collected based on the estimated emotions of the employees. The collection unit estimates the emotions of employees and determines the priority of data to be collected based on the estimated emotions, for example. Emotions include, for example, facial expression analysis, voice analysis, but are not limited thereto. Priority of data includes, for example, importance, urgency, but is not limited thereto. For example, if an employee is feeling stressed, data related to stress is collected preferentially. If the employee is relaxed, data on overall health status can be collected. If the employee is tired, data related to rest or sleep can be collected preferentially. By determining the priority of data to be collected based on employees' emotions, important data can be collected preferentially. Specifically, the collection unit uses multimodal inputs such as facial image data, voice waveform data, and natural language text, and combines convolutional neural networks (CNN), voice emotion classification models, and natural language processing models (BERT series) to estimate emotion labels (e.g., stress, relaxation, fatigue) and emotion scores (e.g., stress level 0.82). Input examples to the AI include 10 facial images, 5 voice clips, and 3 speech texts per day, and output examples include emotion label “stress” and emotion score 0.82. Based on the estimated emotion labels and scores, the collection unit applies a data collection priority judgment algorithm (e.g., stress level>0.7 prioritizes stress-related data, relaxation level>0.7 prioritizes overall health data) to dynamically determine the priority of data to be collected (e.g., stress factor questionnaire, sleep duration, rest status, overall health score). For example, when stress level is high, detailed questionnaires on stress factors and biometric sensor data (e.g., heart rate variability, skin conductance response) are collected preferentially, and when relaxation level is high, overall health data such as dietary habits and exercise routines are collected. These priority controls are personalized by linking with individual employee history databases and are coordinated with past emotion transitions and health status changes. As a technical effect, the collection unit, unlike conventional uniform data collection, can preferentially obtain important data according to employees' psychological state and health risk by combining AI-based emotion estimation and priority control algorithms. As a result, the coverage and reliability of data are improved, and changes in health status and risk signs can be grasped with high accuracy. Specific application fields include health monitoring of field workers where stress management is important, mental health support for office workers, and health maintenance support in remote work environments.

The collection unit can preferentially collect highly relevant data during data collection based on the geographic location information of employees. The collection unit considers employees' geographic location information during data collection and preferentially collects highly relevant data, for example. Geographic location information includes, for example, GPS data, location information services, but is not limited thereto. Highly relevant data includes, for example, location-based related data, but is not limited thereto. For example, if an employee is in the office, data on working hours and work details are collected. If the employee is at home, data on health status and rest can be collected. If the employee is on a business trip, data on movement can be collected. By considering employees' geographic location information and preferentially collecting highly relevant data, more accurate information can be obtained. Specifically, the collection unit acquires GPS coordinate data (e.g., latitude and longitude pairs), Wi-Fi/Bluetooth beacon information, and location labels (e.g., office, home, business trip destination) from location information service APIs in real time from employees' smartphones or wearable devices. The collection unit applies location classification algorithms (e.g., clustering for stay location determination, time-series location estimation) to these location data to accurately identify employees' current location. Input examples to the AI include a series of GPS coordinates for the past 24 hours (e.g., 1440×2 dimensions), Wi-Fi beacon ID list, and location label “office,” and output examples include “current location: office,” “related data collection: working hours, work details,” and “priority: high.” When the current location is the office, the collection unit preferentially collects work-related data such as working hours, work details, and meeting participation status; when at home, it collects data on sleep duration, rest status, and home environment; and when on a business trip, it preferentially acquires data on travel distance, means of transportation, and health status at the business trip destination. These location-linked data collection processes are personalized in conjunction with each employee's work history and health status history. As a technical effect, the collection unit, unlike conventional uniform data collection, can efficiently obtain optimal data according to employees' activity location and work status by combining AI-based location information analysis and related data priority control. As a result, the coverage and reliability of data are improved, and changes in health status and risk signs can be grasped with high accuracy. Specific application fields include safety management of field workers, health monitoring in remote work environments, and health risk management for business trips.

The collection unit can analyze employees' social media activities during data collection and collect relevant data. The collection unit analyzes employees' social media activities during data collection and collects relevant data, for example. Social media activities include, for example, post content, number of likes, but are not limited thereto. Relevant data includes, for example, social media post content, comments, but is not limited thereto. For example, if an employee is feeling stressed on social media, that information is collected. If an employee shares health-related information on social media, that information can also be collected. If an employee shares work-related information on social media, that information can also be collected. By analyzing employees' social media activities, relevant data can be collected. Specifically, the collection unit automatically acquires post text data (e.g., up to 500 characters per post), image data (e.g., 128×128 pixel post images), and interaction information (e.g., number of likes, comments, shares) from major social media platforms used by employees via API. The collection unit applies natural language processing models (e.g., BERT-based emotion analyzers), image analysis models (e.g., CNN for facial expression and atmosphere estimation), and interaction analysis algorithms (e.g., engagement score calculation) to these data to extract “emotion labels” (e.g., stress, health, work-related) and “topic categories” (e.g., health, work, private) from post content. Input examples to the AI include 10 post texts, 5 post images, and time-series data of number of likes and comments for the past week, and output examples include “3 stress-related posts,” “2 health information shares,” and “1 work-related post.” When stress-related posts are detected, the collection unit additionally collects stress factor questionnaires and biometric sensor data; when health information sharing is frequent, it preferentially collects detailed data on health status and lifestyle habits. These social media-linked data collection processes are personalized in conjunction with each employee's history database. As a technical effect, the collection unit, unlike conventional collection limited to self-reporting or business system data, can grasp employees' psychological state and health risks from multiple perspectives and in real time by combining AI-based social media analysis and related data collection algorithms. As a result, changes in health status and risk signs can be detected with high accuracy and early response is possible. Specific application fields include mental health management for office workers, health maintenance support in remote work environments, and stress management for young employees.

The learning unit can estimate the emotions of employees and select learning data based on the estimated emotions of the employees. The learning unit estimates the emotions of employees and selects learning data based on the estimated emotions, for example. Emotions include, for example, facial expression analysis, voice analysis, but are not limited thereto. Selection of learning data includes, for example, selection of data according to changes in emotions, but is not limited thereto. For example, if an employee is feeling stressed, stress-related data is preferentially learned. If the employee is relaxed, data on overall health status can be learned. If the employee is tired, data related to rest or sleep can be learned preferentially. By selecting learning data based on employees' emotions, more effective learning is possible. Specifically, the learning unit uses facial image data (e.g., 128×128 pixel RGB images), voice waveform data (e.g., 16 kHz sampled PCM array), and natural language text (e.g., speech content such as “I'm tired recently”) as input, and combines a facial recognition model using convolutional neural networks (CNN), a voice emotion classification model using spectrogram conversion and recurrent neural networks (RNN), and a BERT-based natural language processing model to estimate “emotion labels” (e.g., stress, relaxation, fatigue) and “emotion scores” (e.g., stress level 0.82, relaxation level 0.15) from each data type. Based on the estimated emotion labels and scores, the learning unit applies a learning data selection algorithm (e.g., stress level>0.7 prioritizes stress-related data, relaxation level>0.7 prioritizes overall health data) to dynamically determine the priority of learning target datasets (e.g., stress factor questionnaire, sleep duration, rest status, overall health score). Input examples to the AI include 10 facial images, 5 voice clips, 3 speech texts, and health status vectors (e.g., 7×10 dimensions) for one week, and output examples include emotion label “stress,” emotion score 0.82, and learning data selection list (e.g., stress factor data prioritized). Based on these outputs, the learning unit personalizes the selection of learning data, focusing on stress factors and coping history when stress level is high, and learning overall health status and lifestyle data when relaxation level is high. Furthermore, when fatigue level is high, the learning unit prioritizes learning data related to sleep and rest to improve the accuracy of detecting changes in health status and risk factors. These processes, unlike conventional uniform data learning, realize optimal learning according to each employee's psychological state and health risk by combining AI-based emotion estimation and dynamic data selection algorithms. As a technical effect, the learning unit improves model personalization accuracy and anomaly detection capability by linking learning data selection to emotional state, enabling early detection of employee health risks and appropriate countermeasure proposals. Specific application fields include call center operations where stress management is important, health monitoring of field workers, and health maintenance support in remote work environments.

The learning unit can determine the level of detail of learning during learning based on the importance of the collected data. The learning unit adjusts the level of detail of learning during learning based on the importance of the collected data, for example. Importance of data includes, for example, data reliability, impact, but is not limited thereto. Level of detail of learning includes, for example, depth of learning according to importance, but is not limited thereto. For example, detailed learning is performed for highly important data. Simplified learning can be performed for less important data. The level of detail of learning can also be dynamically adjusted according to the importance of the data. By adjusting the level of detail of learning based on the importance of the collected data, efficient learning is possible. Specifically, the learning unit applies a data reliability evaluation module (e.g., missing rate of sensor data, consistency score of self-reported data) and an impact estimation algorithm (e.g., contribution analysis to health status change) to health status data (e.g., multidimensional vectors of body temperature, blood pressure, heart rate, sleep duration, stress score) and attendance information (e.g., clock-in/clock-out times, overtime hours, break acquisition status) received from the collection unit, and calculates importance scores for each data point (e.g., reliability 0.95, impact 0.80). Based on these importance scores, the learning unit applies a learning detail control algorithm (e.g., importance>0.8 for detailed learning using multilayer neural networks, importance<0.5 for simplified learning using simple linear models), and dynamically sets different learning depths, number of epochs, and weighting coefficients for each data point. Input examples to the AI include health status vectors for one week (e.g., 7×10 dimensions), reliability score array (e.g., 0.95, 0.80, 0.60, . . . ), and impact score array (e.g., 0.85, 0.70, 0.40, . . . ), and output examples include learning detail setting list (e.g., data 1: detailed learning, data 2: simplified learning), weighting coefficients (e.g., data 1:1.0, data 2:0.5). For highly important data, the learning unit applies deep learning models with multilayer structures and long-term time-series learning to maximize pattern extraction and anomaly detection accuracy. For less important data, the learning unit applies simplified models with reduced computational load or sampling learning to improve overall learning efficiency. These detail controls, unlike conventional uniform learning processing, realize optimal allocation of computational resources and learning accuracy by combining AI-based data importance evaluation and dynamic learning control algorithms. As a technical effect, the learning unit achieves both improved model accuracy and computational efficiency by controlling the level of detail of learning according to data importance, thereby enhancing the reliability of employee health risk prediction and anomaly detection. Specific application fields include health monitoring of field workers where sensor data reliability is an issue, and health management in remote work environments where self-reported data variability is large.

The learning unit can use different learning algorithms during learning according to the category of the data. The learning unit applies different learning algorithms during learning according to the category of the data, for example. Data categories include, for example, health data, attendance data, but are not limited thereto. Learning algorithms include, for example, decision trees, neural networks, but are not limited thereto. For example, health-related algorithms are applied to data on health status. Attendance-related algorithms can be applied to data on attendance information. Work-related algorithms can be applied to data on work details. By applying different learning algorithms according to the category of the data, more accurate learning is possible. Specifically, the learning unit classifies multidimensional data received from the collection unit by category (e.g., health status data, attendance data, work details data, emotion data), and automatically selects the optimal learning algorithm for each category. For health status data (e.g., time-series vectors of body temperature, blood pressure, heart rate, sleep duration), recurrent neural networks (RNN) or long short-term memory networks (LSTM) are applied to learn time-series patterns and abnormal signs. For attendance data (e.g., categorical data of clock-in/clock-out times, overtime hours, break acquisition status), tree-based algorithms such as decision trees or random forests are applied to extract rule-based patterns and detect anomalies. For work details data (e.g., numerical and text-mixed data such as work details, number of meetings attended, workload scores), natural language processing models (e.g., BERT-based classifiers) or multilayer perceptrons (MLP) are applied to extract work patterns and risk factors. Input examples to the AI include health status vectors (7×6 dimensions), attendance data (7×4 dimensions), work details texts (7 items), and emotion scores (7 days), and output examples include health status change score 0.82, attendance anomaly flag 1, and workload label “high.” The learning unit integrates these outputs to detect complex health risks and work anomalies with high accuracy. Furthermore, different loss functions and learning rates are set for each category to optimize the model. These processes, unlike conventional uniform learning with a single algorithm, realize high-precision learning according to data characteristics by combining AI-based category-specific algorithm selection and parameter optimization. As a technical effect, the learning unit improves the reliability of anomaly detection and risk prediction by applying optimal algorithms for each data category, enabling high-precision understanding of complex relationships among health status, attendance, and work patterns. Specific application fields include health management for large organizations integrating multiple data sources, analysis of diverse work patterns for field workers, and complex risk detection in remote work environments.

The learning unit can estimate the emotions of employees and determine the frequency of learning based on the estimated emotions of the employees. The learning unit estimates the emotions of employees and adjusts the frequency of learning based on the estimated emotions, for example. Emotions include, for example, facial expression analysis, voice analysis, but are not limited thereto. Frequency of learning includes, for example, setting learning intervals according to changes in emotions, but is not limited thereto. For example, if an employee is feeling stressed, the frequency of learning is reduced to lessen the burden. If the employee is relaxed, the frequency of learning can be increased to learn more detailed information. If the employee is tired, the frequency of learning can be adjusted to learn at appropriate times. By adjusting the frequency of learning based on employees' emotions, their burden can be reduced. Specifically, the learning unit uses facial image data (e.g., 128×128 pixel facial images), voice waveform data (e.g., 16 kHz sampling), and natural language text (e.g., speech content such as “I'm tired recently”) as input, and combines a CNN-based facial recognition model, an RNN-based voice emotion classification model, and a BERT-based natural language processing model to estimate emotion labels (e.g., stress, relaxation, fatigue) and emotion scores (e.g., stress level 0.78, relaxation level 0.12). Based on the estimated emotion scores, the learning unit applies a learning frequency control algorithm (e.g., stress level>0.7 reduces learning frequency to once a week, relaxation level>0.7 increases learning frequency to twice a day) and dynamically adjusts the learning scheduler. Input examples to the AI include 10 facial images, 5 voice clips, 3 speech texts, and health status vectors (7×10 dimensions) per day, and output examples include emotion label “stress,” emotion score 0.78, and learning frequency “once a week.” When stress level is high, the learning unit reduces the frequency of learning to minimize employees' psychological and physical burden, while increasing the frequency and strengthening learning of detailed health status and behavioral patterns when relaxation level is high. Furthermore, when fatigue level is high, the learning unit automatically adjusts the timing of learning to after work or during breaks, when employee burden is low. These frequency controls, unlike conventional fixed-interval learning, realize optimal learning frequency according to each employee's psychological state and health risk by combining AI-based emotion estimation and dynamic scheduling algorithms. As a technical effect, the learning unit achieves both learning efficiency and reduction of employee burden by personalized control of learning frequency, contributing to early detection of health risks and improved work efficiency. Specific application fields include call center operations where stress management is important, health monitoring of field workers, and health maintenance support in remote work environments.

The learning unit can weight learning data during learning based on the timing of data collection. The learning unit weights learning data during learning based on the timing of data collection, for example. Timing of data collection includes, for example, timestamps, collection dates and times, but is not limited thereto. Weighting includes, for example, giving higher priority to the latest data, but is not limited thereto. For example, higher weighting is given to recently collected data. Lower weighting can be given to older data. Weighting can also be dynamically adjusted according to the timing of data collection. By weighting learning data based on the timing of data collection, learning that emphasizes the latest information is possible. Specifically, the learning unit uses timestamps (e.g., 2024-06-01 09:00:00) attached to health status data and attendance information received from the collection unit, and applies data weighting algorithms (e.g., exponential decay function w=exp(−λ×elapsed days), linear decay w=max(0,1-α×elapsed days)) to assign higher weights to more recent data. Input examples to the AI include health status vectors for one month (30×10 dimensions), timestamp array for each data point (e.g., 30 items), and elapsed days array (e.g., 0,1,2, . . . ,29 days), and output examples include weighting coefficient array (e.g., latest data 1.0, 1 day ago 0.95, 7 days ago 0.80, 30 days ago 0.50). The learning unit reflects these weighting coefficients in the loss function and gradient calculation during learning, and optimizes model parameters with emphasis on the latest health status and attendance changes. Furthermore, the parameters of the weighting function (λ and α) are automatically adjusted according to the data variation characteristics of each industry and employee. These weighting processes, unlike conventional uniform learning, realize optimal balance between tracking the latest information and utilizing past data by combining AI-based time-series data weighting and dynamic parameter control. As a technical effect, the learning unit improves the real-time performance of anomaly detection and risk prediction by quickly and accurately reflecting the latest changes in health status and attendance through weighting based on timing of data collection. Specific application fields include monitoring of field workers where sudden changes in health status are a problem, attendance anomaly detection in remote work environments, and health risk management during project progress.

The learning unit can determine the order of learning during learning based on the relevance of the data. The learning unit adjusts the order of learning during learning based on the relevance of the data, for example. Relevance of data includes, for example, correlation analysis, relevance scores, but is not limited thereto. Order of learning includes, for example, priority of learning based on relevance, but is not limited thereto. For example, data with high relevance is learned preferentially. Data with low relevance can be postponed. The order of learning can also be dynamically adjusted according to the relevance of the data. By adjusting the order of learning based on the relevance of the data, efficient learning is possible. Specifically, the learning unit applies correlation analysis algorithms (e.g., Pearson correlation coefficient, Spearman rank correlation) and relevance score calculation (e.g., mutual information, graph-based relevance) to multidimensional data (e.g., health status vectors, attendance information, work details data, emotion scores) received from the collection unit, and calculates relevance scores for each data pair (e.g., correlation between health status and attendance: 0.85, correlation between work details and emotion scores: 0.65). Based on these relevance scores, the learning unit applies a learning order control algorithm (e.g., learning in descending order of relevance scores, postponing those below a threshold) and dynamically generates a priority list for learning data. Input examples to the AI include health status vectors for one month (30×10 dimensions), attendance data (30×4 dimensions), work details data (30 items), and emotion scores (30 days), and output examples include learning order list (e.g., health status→attendance→work details→emotion), relevance score array (e.g., 0.85, 0.75, 0.65, 0.40). By prioritizing learning from highly relevant data, the learning unit improves the efficiency of pattern extraction and anomaly detection accuracy of the model. By postponing learning from less relevant data, the learning unit optimizes allocation of computational resources and learning efficiency. Furthermore, relevance scores are recalculated periodically, and the order of learning is automatically updated according to data variation and addition of new data. These order controls, unlike conventional random or uniform order learning, realize efficient and high-precision learning by combining AI-based relevance evaluation and dynamic order optimization. As a technical effect, the learning unit improves both the accuracy and computational efficiency of anomaly detection and risk prediction by controlling the order of learning based on data relevance, enabling efficient understanding of complex relationships among health status, attendance, and work patterns. Specific application fields include health management for large organizations integrating multiple data sources, analysis of diverse work patterns for field workers, and complex risk detection in remote work environments.

The detection unit can estimate the emotions of employees and determine the criteria for detecting abnormal values based on the estimated emotions of the employees. The detection unit estimates the emotions of employees and adjusts the criteria for detecting abnormal values based on the estimated emotions, for example. Emotions include, for example, facial expression analysis, voice analysis, but are not limited thereto. Criteria for detecting abnormal values include, for example, setting thresholds according to changes in emotions, but are not limited thereto. For example, if an employee is feeling stressed, the criteria for detecting abnormal values are made stricter. If the employee is relaxed, the criteria for detecting abnormal values can be relaxed. If the employee is tired, the criteria for detecting abnormal values can be adjusted. By adjusting the criteria for detecting abnormal values based on employees' emotions, more accurate detection of abnormal values is possible. Specifically, the detection unit uses facial image data (e.g., 128×128 pixel facial images), voice waveform data (e.g., 16 kHz sampled PCM array), and natural language text (e.g., speech content such as “I'm tired recently”) as input, and combines a CNN-based facial recognition model, a voice emotion classification model using spectrogram conversion and RNN, and a BERT-based natural language processing model to estimate “emotion labels” (e.g., stress, relaxation, fatigue) and “emotion scores” (e.g., stress level 0.82, relaxation level 0.15) from each data type. Based on the estimated emotion scores, the detection unit applies an abnormal value detection criteria control algorithm (e.g., stress level>0.7 sets the abnormal threshold to 0.6 for stricter detection, relaxation level>0.7 relaxes the threshold to 0.8) and dynamically adjusts the thresholds for judging abnormality in health status change scores and biometric indicators. Input examples to the AI include 10 facial images, 5 voice clips, 3 speech texts, and a series of health status change scores (e.g., 0.65, 0.68, 0.82, . . . ) per day, and output examples include emotion label “stress,” emotion score 0.82, and abnormal value detection threshold 0.6. When stress level is high, the detection unit makes the criteria for judging abnormality in health status change scores, heart rate, sleep duration, etc. stricter, and detects even subtle changes as abnormal. When relaxation level is high, the detection unit relaxes the thresholds to suppress excessive alerts. Furthermore, when fatigue level is high, the detection unit finely adjusts the thresholds for specific indicators (e.g., sleep duration, rest status) according to the emotional state. These processes, unlike conventional fixed-threshold anomaly detection, realize optimal anomaly detection according to each employee's psychological state and health risk by combining AI-based multimodal emotion estimation and dynamic threshold control algorithms. As a technical effect, the detection unit improves both the accuracy and real-time performance of anomaly detection by dynamically adjusting the criteria for detecting abnormal values in conjunction with emotional state, enabling early detection of employee health risks and suppression of excessive alerts. Specific application fields include call center operations where stress management is important, safety monitoring of field workers, and health maintenance support in remote work environments.

The detection unit can improve the accuracy of abnormal value detection based on the interrelationships of data during detection. For example, the detection unit improves the accuracy of abnormal value detection by considering the interrelationships of data during detection. The interrelationships of data may include, for example, correlation analysis and co-occurrence networks, but are not limited thereto. The accuracy of abnormal value detection may include, for example, methods for improving accuracy based on interrelationships, but is not limited thereto. For example, abnormal values are detected by considering the interrelationship between health status and attendance information. Abnormal values can also be detected by considering the interrelationship between health status and work details. Furthermore, abnormal values can be detected by considering the interrelationship between attendance information and work details. By considering the interrelationships of data, the accuracy of abnormal value detection is improved. Specifically, the detection unit inputs multidimensional data such as health status data (e.g., time-series vectors of body temperature, blood pressure, heart rate, sleep duration), attendance data (e.g., clock-in/out times, overtime hours, break status), and work details data (e.g., work content, number of meetings attended, workload score), and applies correlation analysis algorithms (e.g., Pearson correlation coefficient, Spearman rank correlation), co-occurrence network analysis (e.g., co-occurrence frequency graphs between features), and multivariate anomaly detection models (e.g., principal component analysis-based anomaly scoring, autoencoder-based multidimensional reconstruction error). The detection unit calculates interrelationship scores between each data category (e.g., correlation of 0.85 between health status and attendance, correlation of 0.65 between work details and health status), and dynamically adjusts the weighting and threshold settings of the abnormal value detection algorithm based on these scores. Examples of AI input include one month of health status vectors (30×10 dimensions), attendance data (30×4 dimensions), and work details data (30 items), and examples of output include anomaly detection flag 1, anomaly score 0.88, and interrelationship score array (e.g., 0.85, 0.65, 0.70). When a strong correlation between health status and attendance information is observed, the detection unit strengthens anomaly judgment by focusing on simultaneous anomalies (e.g., increased heart rate+increased overtime). On the other hand, when the correlation between work details and health status is low, independent anomaly detection is performed, enabling flexible judgment according to data characteristics. Furthermore, co-occurrence network analysis automatically extracts simultaneous anomaly patterns of multiple features (e.g., decreased sleep duration+increased workload+increased heart rate), enabling highly accurate composite risk detection. Unlike conventional single-feature-based anomaly detection, these processes combine AI-based multivariate correlation analysis and network structure analysis to detect anomalies caused by complex work and health patterns with high accuracy and at an early stage. As a technical effect, the detection unit, by applying anomaly detection algorithms based on data interrelationships, greatly reduces false positives and missed detections, contributing to organization-wide health risk management and operational efficiency. Specific application fields include health management for large organizations integrating multiple data sources, analysis of diverse work patterns for field workers, and composite risk detection in remote work environments, and can be deployed in various industries and occupations.

The detection unit can detect abnormal values based on employee attribute information during detection. For example, the detection unit detects abnormal values by considering employee attribute information during detection. Attribute information may include, for example, age, gender, and job type, but is not limited thereto. Abnormal values may include, for example, degree of deviation from the normal range and abnormal value thresholds, but are not limited thereto. For example, abnormal values are detected by considering the age and gender of employees. Abnormal values can also be detected by considering the job type and position of employees. Furthermore, abnormal values can be detected by considering the years of service and experience of employees. By considering employee attribute information, more accurate abnormal value detection becomes possible. Specifically, the detection unit receives as input health status data (e.g., body temperature, blood pressure, heart rate, sleep duration), attendance data (e.g., clock-in/out times, overtime hours), work details data (e.g., work content, number of meetings attended), and employee attribute information (e.g., age 35, male, engineer, manager, 10 years of service). The detection unit refers to an attribute-specific normal range database (e.g., age group-specific normal heart rate range, job type-specific overtime standards, position-specific stress tolerance) based on attribute information, and automatically sets individualized abnormal value judgment thresholds. Examples of AI input include health status vectors (7×6 dimensions), attendance data (7×4 dimensions), and attribute information vectors (age, gender, job type, position, years of service), and examples of output include anomaly detection flag 1, anomaly score 0.90, and anomaly factor label “exceeded age group standard.” For example, strict standards for heart rate and stress indicators are applied to younger employees, while standards for blood pressure and physical condition scores are adjusted for middle-aged and older employees. In addition, abnormal judgment standards for overtime hours and workload are dynamically changed according to job type and position, and standards are corrected based on years of service and experience. Unlike conventional uniform abnormal value judgment, these processes apply AI-based attribute-linked individualized anomaly detection algorithms, enabling highly accurate anomaly detection optimized for each employee's characteristics and risk profile. As a technical effect, the detection unit, by personalizing abnormal value judgment based on attribute information, greatly reduces false positives and missed detections, enabling early detection of health risks and appropriate countermeasure proposals. Specific application fields include health management for large organizations with diverse ages, genders, and job types, attribute-based risk management for field workers, and individually optimized health monitoring in remote work environments, and can be deployed in various industries and occupations.

The detection unit can estimate the emotions of employees and determine the display method of abnormal values based on the estimated emotions of the employees. For example, the detection unit estimates the emotions of employees and adjusts the display method of abnormal values based on the estimated emotions of the employees. Emotions may include, for example, facial expression analysis and voice analysis, but are not limited thereto. Display methods for abnormal values may include, for example, setting display formats according to changes in emotions, but are not limited thereto. For example, when an employee is feeling stressed, a simple and highly visible display method is provided. When an employee is relaxed, a display method including detailed information can also be provided. When an employee is in a hurry, a display method focusing on key points can also be provided. By adjusting the display method of abnormal values based on the emotions of employees, highly visible displays become possible. Specifically, the detection unit inputs facial image data (e.g., 128×128 pixel face images), voice waveform data (e.g., 16 kHz sampling), and natural language text (e.g., utterances such as “I am in a hurry”), and combines a CNN-based facial expression recognition model, an RNN-based voice emotion classification model, and a BERT-based natural language processing model to estimate emotion labels (e.g., stress, relaxation, fatigue, tension) and emotion scores (e.g., stress level 0.78, relaxation level 0.12, tension level 0.65). Based on the estimated emotion scores, the detection unit applies an abnormal value display control algorithm (e.g., simple display for stress level>0.7, detailed display for relaxation level>0.7, key point emphasis display for tension level>0.6), and dynamically adjusts the display layout, information volume, and highlight colors of dashboards and notification screens. Examples of AI input include 10 facial images per day, 5 voice clips, 3 utterance texts, anomaly detection flag 1, and anomaly score 0.85, and examples of output include display format “simple,” highlight item “anomaly score,” and notification message “Caution: High stress level.” When the stress level is high, the detection unit provides a simple display that emphasizes only the abnormal value, minimizing employee burden and confusion. When the relaxation level is high, a rich display including detailed breakdowns of abnormal values, past trend graphs, and countermeasures is provided. Furthermore, when the tension or urgency level is high, only key points are prominently displayed to support quick decision-making. Unlike conventional fixed layouts or uniform information displays, these display controls combine AI-based emotion estimation and dynamic UI optimization algorithms to provide optimal information presentation tailored to each employee's psychological state and work situation. As a technical effect, the detection unit, by controlling abnormal value display in conjunction with emotional states, maximizes information visibility, comprehension, and action inducement, contributing to health risk response and operational efficiency. Specific application fields include health monitoring for field workers where stress management is important, mental health support for office workers, and health maintenance support in remote work environments, and can be deployed in various industries and occupations.

The detection unit can detect abnormal values based on the geographic distribution of data during detection. For example, the detection unit detects abnormal values by considering the geographic distribution of data during detection. Geographic distribution may include, for example, map data and location information services, but is not limited thereto. Abnormal values may include, for example, degree of deviation from the normal range and abnormal value thresholds, but are not limited thereto. For example, abnormal values are detected based on the employee's work location. Abnormal values can also be detected based on the employee's business trip destination. Furthermore, abnormal values can be detected based on the employee's home working location. By considering the geographic distribution of data, the accuracy of abnormal value detection is improved. Specifically, the detection unit inputs GPS coordinate data (e.g., latitude and longitude pairs) obtained from employees' smartphones and wearable devices, Wi-Fi/Bluetooth beacon information, and location labels from location information service APIs (e.g., office, home, business trip destination), and applies location classification algorithms (e.g., clustering-based stay location determination, time-series location estimation) to accurately identify the employee's current location and movement history. The detection unit refers to a location-specific abnormal value judgment criteria database (e.g., normal heart rate range during office work, stress criteria during home work, travel distance criteria during business trips) and automatically selects the optimal abnormal value detection algorithm for each location. Examples of AI input include GPS coordinate sequences for the past week (e.g., 10080×2 dimensions), location label arrays (e.g., office, home, business trip destination), and health status vectors (7×10 dimensions), and examples of output include anomaly detection flag 1, anomaly score 0.92, and anomaly factor label “exceeded business trip destination standard.” When the work location is the office, the detection unit emphasizes abnormal judgment based on work-related data (e.g., working hours, meeting participation status), and when working from home, emphasizes indicators of lifestyle rhythm such as sleep duration and rest status. During business trips, the detection unit focuses on monitoring travel distance and changes in health status at the business trip destination, and dynamically adjusts abnormal value detection criteria. Unlike conventional uniform abnormal value judgment, these processes combine AI-based geographic distribution analysis and location-linked anomaly detection algorithms to achieve highly accurate anomaly detection tailored to the employee's activity location and work situation. As a technical effect, the detection unit, by optimizing abnormal value judgment based on geographic distribution, contributes to early detection of health risks, operational efficiency, and safety management of business trips. Specific application fields include safety management for field workers, health monitoring in remote work environments, and health risk management for business trips, and can be deployed in various industries and occupations.

The detection unit can improve the accuracy of abnormal value detection based on related literature during detection. For example, the detection unit improves the accuracy of abnormal value detection by referring to related literature during detection. Related literature may include, for example, academic papers and technical reports, but is not limited thereto. The accuracy of abnormal value detection may include, for example, methods for improving accuracy based on literature, but is not limited thereto. For example, abnormal values are detected by referring to the latest research on health status. Abnormal values can also be detected by referring to the latest research on attendance information. Furthermore, abnormal values can be detected by referring to the latest research on work details. By referring to related literature, the accuracy of abnormal value detection is improved. Specifically, the detection unit inputs health status data (e.g., body temperature, blood pressure, heart rate, sleep duration), attendance data (e.g., clock-in/out times, overtime hours), and work details data (e.g., work content, number of meetings attended), and refers to a related literature database (e.g., latest papers on health indicator standards, guidelines for abnormal attendance judgment, reports on workload evaluation) to automatically extract reference values, judgment rules, and abnormal patterns, and dynamically optimize the parameters and thresholds of the abnormal value detection algorithm. Examples of AI input include health status vectors (7×10 dimensions), attendance data (7×4 dimensions), work details data (7 items), and literature reference value lists (e.g., normal heart rate range 60-100 bpm, overtime standard 40 hours/month), and examples of output include anomaly detection flag 1, anomaly score 0.88, and reference literature ID “2024-Health-001.” The detection unit automatically updates reference values according to the employee's age, gender, and job type by referring to the latest research on health status, and performs abnormal judgment reflecting the latest guidelines on attendance information and work details. Furthermore, literature information is regularly updated, and when new health risks or abnormal work patterns are discovered, they are immediately reflected in the anomaly detection algorithm. Unlike conventional anomaly detection relying on static reference values or empirical rules, these processes combine AI-based automatic literature referencing and parameter optimization algorithms to achieve highly accurate anomaly detection based on the latest scientific knowledge. As a technical effect, the detection unit, by literature-referenced abnormal value judgment, prevents missed health risks and work anomalies, contributing to improved safety and productivity for the entire organization. Specific application fields include industries where reflecting the latest research results is important, such as medical, manufacturing, and IT, health management for field workers where guideline compliance is required, and anomaly detection in remote work environments, and can be deployed in various industries and occupations.

The guidance unit can estimate the emotions of employees and determine the priority of treatment or consultation destinations to be guided based on the estimated emotions of the employees. For example, the guidance unit estimates the emotions of employees and determines the priority of treatment or consultation destinations to be guided based on the estimated emotions of the employees. Emotions may include, for example, facial expression analysis and voice analysis, but are not limited thereto. The priority of treatment or consultation destinations may include, for example, setting priorities according to changes in emotions, but is not limited thereto. For example, when an employee is feeling stressed, a stress management expert is prioritized for guidance. When an employee is relaxed, a general health management expert can be guided. When an employee is fatigued, a rest or sleep expert can be prioritized for guidance. By determining the priority of treatment or consultation destinations based on the emotions of employees, more appropriate guidance becomes possible. Specifically, the guidance unit inputs facial image data (e.g., 128×128 pixel face images), voice waveform data (e.g., 16 kHz sampled PCM array), and natural language text (e.g., utterances such as “I have been tired recently”), and combines a convolutional neural network (CNN)-based facial expression recognition model, spectrogram conversion+recurrent neural network (RNN)-based voice emotion classification model, and BERT-based natural language processing model to estimate emotion labels (e.g., stress, relaxation, fatigue) and emotion scores (e.g., stress level 0.82, relaxation level 0.15). Based on the estimated emotion labels and scores, the guidance unit applies a guidance priority determination algorithm (e.g., prioritize stress management expert for stress level>0.7, prioritize health management expert for relaxation level>0.7, prioritize rest/sleep expert for fatigue level>0.7), and assigns priorities to the list of treatment proposals or consultation destinations (e.g., occupational physician interview, stress check, recommendation for taking leave, in-house counselor, external consultation desk). Examples of AI input include 10 facial images per day, 5 voice clips, 3 utterance texts, health status vectors (7×10 dimensions), and examples of output include emotion label “stress,” emotion score 0.82, and guidance priority list (e.g., 1st: stress management expert, 2nd: rest recommendation, 3rd: health management expert). Based on these outputs, the guidance unit automatically notifies the employee or supervisor of priority guidance messages (e.g., “Since your stress level is currently high, we will guide you to consult a stress management expert as the highest priority”) via chat tool API or dashboard. Furthermore, the guidance priority is dynamically adjusted in conjunction with each employee's history database according to past consultation history and health status trends. As a technical effect, unlike conventional uniform guidance or subjective judgment, the guidance unit combines AI-based multimodal emotion estimation and priority control algorithms to achieve highly accurate and real-time optimal guidance for treatment or consultation destinations according to the employee's psychological state and health risk. As a result, it contributes to early detection of health risks and prompt response, reduction of employee burden, and operational efficiency. Specific application fields include call center operations where stress management is important, health monitoring for field workers, and mental health support in remote work environments, and can be deployed in various industries and occupations.

The guidance unit can analyze employees' past health status or attendance information during guidance and select appropriate treatment or consultation destinations. For example, the guidance unit analyzes employees' past health status or attendance information during guidance and selects optimal treatment or consultation destinations. Past health status may include, for example, past diagnosis results and health checkup data, but is not limited thereto. Attendance information may include, for example, clock-in times, clock-out times, and break times, but is not limited thereto. For example, the guidance unit analyzes employees' past health status and guides them to experts if their health status is deteriorating. The guidance unit can also analyze attendance information and propose rest if working hours are long. Furthermore, the guidance unit can analyze work details and guide employees to stress management experts if workload is high. By analyzing employees' past health status or attendance information, optimal treatment or consultation destinations can be selected. Specifically, the guidance unit obtains each employee's past one year of health checkup data (e.g., time-series vectors of body temperature, blood pressure, heart rate, blood test values), attendance history (e.g., time-series tensors of clock-in/out times, overtime hours, break status), and work report data (e.g., work details, workload score) from the database. The guidance unit inputs these multidimensional time-series data and applies anomaly detection algorithms (e.g., autoencoder-based anomaly scoring, time-series clustering) and pattern mining methods (e.g., frequent pattern extraction, trend analysis) to automatically extract health deterioration trends (e.g., continuous decrease in physical condition score, increasing overtime hours) and attendance anomalies (e.g., frequent lateness/early leave). Examples of AI input include 30 days of physical condition score series (e.g., 30×3 dimensions), attendance history (e.g., 30×4 dimensions), and workload score (e.g., continuous values for 30 days), and examples of output include “health deterioration flag 1,” “recommendation for detailed data collection,” and guidance destination list (e.g., occupational physician, counselor, recommendation for rest). Based on these outputs, the guidance unit prioritizes occupational physician interviews and counseling services for employees with detected health deterioration, and guides employees to rest or stress management experts if attendance anomalies or increased workload are detected. Furthermore, the guidance content is dynamically optimized in conjunction with each employee's history database according to past consultation history and health status trends. As a technical effect, unlike conventional uniform guidance or subjective judgment, the guidance unit combines AI-based past data analysis and dynamic guidance destination selection algorithms to achieve highly accurate and real-time optimal guidance for treatment or consultation destinations according to each employee's health risk and work situation. As a result, it contributes to early detection of health risks and prompt response, reduction of employee burden, and operational efficiency. Specific application fields include the IT industry where long working hours are a problem, health monitoring for field workers, and attendance anomaly detection for shift workers, and can be deployed in various industries and occupations.

The guidance unit can customize treatment or consultation destinations during guidance based on employees' current living conditions. For example, the guidance unit customizes treatment or consultation destinations during guidance based on employees' current living conditions. Living conditions may include, for example, lifestyle habits and family environment, but are not limited thereto. For example, when an employee is feeling stressed due to family circumstances, a family problem expert is guided. When an employee has health problems, a medical expert can be guided. Furthermore, when an employee is fatigued due to work load, a relaxation expert can be guided. By customizing treatment or consultation destinations based on employees' current living conditions, more appropriate guidance becomes possible. Specifically, the guidance unit receives as input lifestyle habit questionnaire data entered by employees (e.g., sleep duration, meal content, exercise frequency), family environment information (e.g., family composition, family stress factors), and workload score (e.g., work volume index, overtime hours). Based on these data, the guidance unit applies lifestyle condition classification algorithms (e.g., clustering-based lifestyle pattern classification, family environment stress estimation model) and risk factor extraction models (e.g., BERT-based text classifier) to comprehensively evaluate employees' current living conditions. Examples of AI input include one week of lifestyle habit data (e.g., 7×5 dimensions), family environment questionnaire (e.g., 5 items), and workload score (e.g., continuous values for 7 days), and examples of output include “living condition label: family stress,” “recommended guidance destination: family problem expert,” and “guidance content: family stress consultation service.” According to the living condition label, the guidance unit automatically selects the optimal guidance destination from family problem experts, medical experts, relaxation experts, health management advisors, etc., and notifies the employee or supervisor of customized guidance messages via chat tool API or dashboard. Furthermore, the guidance content is dynamically optimized in conjunction with each employee's history database according to past living conditions and consultation history. As a technical effect, unlike conventional uniform guidance or subjective judgment, the guidance unit combines AI-based living condition analysis and customized guidance algorithms to achieve highly accurate and real-time optimal guidance for treatment or consultation destinations according to each employee's individual circumstances and risk factors. As a result, it contributes to early detection of health risks and prompt response, reduction of employee burden, and operational efficiency. Specific application fields include health management for field workers whose work is affected by family problems, health support for office workers at high risk of lifestyle-related diseases, and lifestyle rhythm optimization in remote work environments, and can be deployed in various industries and occupations.

The guidance unit can estimate the emotions of employees and determine the display method of treatment or consultation destinations to be guided based on the estimated emotions of the employees. For example, the guidance unit estimates the emotions of employees and adjusts the display method of treatment or consultation destinations to be guided based on the estimated emotions of the employees. Emotions may include, for example, facial expression analysis and voice analysis, but are not limited thereto. Display methods may include, for example, setting display formats according to changes in emotions, but are not limited thereto. For example, when an employee is feeling stressed, a simple and highly visible display method is provided. When an employee is relaxed, a display method including detailed information can also be provided. When an employee is in a hurry, a display method focusing on key points can also be provided. By adjusting the display method based on the emotions of employees, highly visible guidance becomes possible. Specifically, the guidance unit inputs facial image data (e.g., 128×128 pixel face images), voice waveform data (e.g., 16 kHz sampling), and natural language text (e.g., utterances such as “I am in a hurry”), and combines a CNN-based facial expression recognition model, an RNN-based voice emotion classification model, and a BERT-based natural language processing model to estimate emotion labels (e.g., stress, relaxation, fatigue, tension) and emotion scores (e.g., stress level 0.78, relaxation level 0.12, tension level 0.65). Based on the estimated emotion scores, the guidance unit applies a guidance display control algorithm (e.g., simple display for stress level>0.7, detailed display for relaxation level>0.7, key point emphasis display for tension level>0.6), and dynamically adjusts the display layout, information volume, and highlight colors of dashboards and notification screens. Examples of AI input include 10 facial images per day, 5 voice clips, 3 utterance texts, guidance content list (e.g., stress management expert guidance, health management guidance), and examples of output include display format “simple,” highlight item “priority guidance destination,” and notification message “Caution: High stress level.” When the stress level is high, the guidance unit provides a simple display that emphasizes only the guidance destination, minimizing employee burden and confusion. When the relaxation level is high, a rich display including detailed breakdowns of guidance destinations, past trend graphs, and countermeasures is provided. Furthermore, when the tension or urgency level is high, only key points are prominently displayed to support quick decision-making. Unlike conventional fixed layouts or uniform information displays, these display controls combine AI-based emotion estimation and dynamic UI optimization algorithms to provide optimal information presentation tailored to each employee's psychological state and work situation. As a technical effect, the guidance unit, by controlling guidance display in conjunction with emotional states, maximizes information visibility, comprehension, and action inducement, contributing to health risk response and operational efficiency. Specific application fields include health monitoring for field workers where stress management is important, mental health support for office workers, and health maintenance support in remote work environments, and can be deployed in various industries and occupations.

The guidance unit can select appropriate treatment or consultation destinations during guidance based on employees' geographic location information. For example, the guidance unit selects optimal treatment or consultation destinations during guidance by considering employees' geographic location information. Geographic location information may include, for example, GPS data and location information services, but is not limited thereto. For example, when an employee is in the office, nearby experts are guided. When an employee is at home, online consultation destinations can be guided. When an employee is on a business trip, experts at the business trip destination can be guided. By considering employees' geographic location information, optimal treatment or consultation destinations can be guided. Specifically, the guidance unit obtains GPS coordinate data (e.g., latitude and longitude pairs), Wi-Fi/Bluetooth beacon information, and location labels (e.g., office, home, business trip destination) from location information service APIs in real time from employees' smartphones and wearable devices. The guidance unit inputs these location information and applies location classification algorithms (e.g., clustering-based stay location determination, time-series location estimation) to accurately identify the employee's current location. Examples of AI input include GPS coordinate sequences for the past 24 hours (e.g., 1440×2 dimensions), Wi-Fi beacon ID list, location label “office,” and examples of output include “current location: office,” “guidance destination list: nearby experts,” and “guidance method: face-to-face.” When the current location is the office, the guidance unit prioritizes guidance to nearby occupational physicians and counselors, and when at home, guides online consultation services and remote counseling. During business trips, the guidance unit guides medical institutions and local experts at the business trip destination. These location-linked guidance are personalized in conjunction with each employee's work history and health status history. As a technical effect, unlike conventional uniform guidance, the guidance unit combines AI-based location information analysis and related guidance destination selection algorithms to efficiently achieve optimal guidance for treatment or consultation destinations according to the employee's activity location and work situation. As a result, it contributes to early detection of health risks, operational efficiency, and safety management of business trips. Specific application fields include safety management for field workers, health monitoring in remote work environments, and health risk management for business trips, and can be deployed in various industries and occupations.

The guidance unit can analyze employees' social media activities during guidance and propose treatment or consultation destinations. For example, the guidance unit analyzes employees' social media activities during guidance and proposes treatment or consultation destinations. Social media activities may include, for example, post content and number of likes, but are not limited thereto. For example, when an employee is feeling stressed on social media, experts are guided based on that information. When an employee shares health-related information on social media, experts can also be guided based on that information. Furthermore, when an employee shares work-related information on social media, experts can also be guided based on that information. By analyzing employees' social media activities, more appropriate treatment or consultation destinations can be proposed. Specifically, the guidance unit automatically obtains post text data (e.g., up to 500 characters per post), image data (e.g., 128×128 pixel post images), and interaction information (e.g., number of likes, comments, shares) from major social media platforms used by employees via API. The guidance unit inputs these data and applies natural language processing models (e.g., BERT-based emotion analyzer), image analysis models (e.g., CNN-based facial expression and atmosphere estimation), and interaction analysis algorithms (e.g., engagement score calculation) to extract emotion labels (e.g., stress, health, work-related) and topic categories (e.g., health, work, private) from post content. Examples of AI input include 10 post texts from the past week, 5 post images, and time-series data of number of likes and comments, and examples of output include “3 stress-related posts,” “2 health information sharing posts,” “1 work-related post,” and recommended guidance destination list (e.g., stress management expert, health management advisor). When stress-related posts are detected, the guidance unit prioritizes guidance to stress management experts and counseling services, and when health information sharing is frequent, guides health management advisors and lifestyle improvement services. When work-related posts are frequent, the guidance unit guides workload management experts and occupational physicians. These social media-linked guidance are personalized in conjunction with each employee's history database. As a technical effect, unlike conventional guidance limited to self-reporting or business system data, the guidance unit combines AI-based social media analysis and related guidance destination selection algorithms to comprehensively and in real time grasp employees' psychological states and health risks, and achieve highly accurate guidance for treatment or consultation destinations. As a result, changes in health status and risk signs can be detected early, enabling prompt response. Specific application fields include mental health management for office workers, health maintenance support in remote work environments, and stress management for young employees, and can be deployed in various industries and occupations.

The system according to the embodiment is not limited to the examples described above, and various modifications are possible, for example, as follows. Specifically, the system has extensibility that allows flexible changes to data input formats, AI model configurations, algorithm selection, and data flow control through modular design of each component such as the collection unit, learning unit, detection unit, and guidance unit. For example, the system can add data acquisition functions from new sensor devices (e.g., skin conductance sensors, activity trackers, sleep trackers) to the collection unit, or incorporate the latest large language models and graph neural networks into the learning unit. In the detection unit, multiple AI architectures such as autoencoders, variational autoencoders, and LSTM-based models for time-series anomaly detection can be selectively applied as anomaly detection algorithms. In the guidance unit, flexible guidance methods can be realized according to operational requirements, such as customization of chatbot APIs and dashboard UIs, and expansion of notification destinations (e.g., employee, supervisor, occupational physician, family). Furthermore, the system allows various configuration variations according to actual operational environments, such as distributed processing on cloud environments or edge devices, multi-site and multilingual support, and API integration with external health management systems or attendance management systems. With such flexible modifiability and extensibility, the system exhibits the technical effect of being able to quickly adapt to technological evolution and changes in operational requirements, unlike conventional fixed health management systems. As a result, it is possible to build optimal health and attendance management solutions according to the scale, industry, and operational policies of the organization, contributing to long-term reduction of system operation costs and maximization of implementation effects. Specific application fields include safety management for field workers in manufacturing, health support for remote work in IT companies, and multi-professional collaborative health monitoring in medical institutions, and can be deployed in various industries, occupations, and operational forms.

The collection unit can collect not only employees' health status and attendance information, but also data on employees' dietary habits and exercise routines. For example, the collection unit collects information on employees' daily dietary habits and exercise routines, which is useful for understanding health status. The collection unit can also collect data from wearable devices to measure employees' sleep quality. This enables a more comprehensive understanding of employees' health status. Furthermore, the collection unit can collect responses to stress check sheets to measure employees' stress levels, which is useful for stress management. Specifically, the collection unit collects health status data (e.g., multidimensional vectors of body temperature, blood pressure, heart rate, blood test values), attendance information (e.g., time-series tensors of clock-in/out times, overtime hours, break status), dietary data (e.g., food names, quantities, calories, nutrient vectors for three meals per day), exercise routine data (e.g., step count, exercise type, exercise duration, calories burned), sleep data (e.g., sleep duration, percentage of deep sleep, number of awakenings, sleep score), and stress check sheet responses (e.g., five-point scale scores for 10 items). Examples of AI input include one week of meal records (7×3 meals×10 items), exercise records (exercise type, duration, calories burned for 7 days), sleep data (sleep scores for 7 days), and stress check responses (10 items). The collection unit integrates these diverse data and utilizes them for multifaceted understanding of health status and early detection of lifestyle risk. For example, the collection unit automatically detects dietary imbalances, lack of exercise, decline in sleep quality, and increase in stress scores, and provides highly accurate input data to subsequent learning and detection units. Unlike conventional single-faceted health and attendance data collection, these processes combine AI-based multimodal data integration and lifestyle analysis to enable comprehensive understanding and individual optimization of health risks, exhibiting technical effects. Specific application fields include health support for office workers at high risk of lifestyle-related diseases, safety management for field workers, and lifestyle rhythm optimization in remote work environments, and can be deployed in various industries and occupations.

The learning unit can perform personalized learning based on individual employee characteristics when analyzing collected information. For example, the learning unit detects changes in health status more accurately by considering employee attribute information such as age, gender, and job type. The learning unit can also predict future health risks based on employees' past health data and attendance information, enabling employees to take preventive measures against health risks. Furthermore, the learning unit can propose optimal health management plans based on employees' lifestyle habits and work patterns, which is useful for maintaining employee health. Specifically, the learning unit integrates and inputs health status data (e.g., multidimensional vectors of body temperature, blood pressure, heart rate, sleep duration), attendance information (e.g., clock-in/out times, overtime hours, break status), attribute information (e.g., age, gender, job type, position, years of service), lifestyle data (e.g., dietary habits, exercise frequency, sleep score), and work patterns (e.g., shift work, flextime, remote work) received from the collection unit. Based on these data, the learning unit combines attribute-embedded personalized neural networks (e.g., attribute embedding+multilayer perceptron), time-series prediction models (e.g., LSTM, GRU), and clustering algorithms (e.g., K-means, hierarchical clustering) to perform optimized detection of health status changes and health risk prediction for each employee. Examples of AI input include one year of health status vectors (365×10 dimensions), attendance history (365×4 dimensions), attribute information vectors (5 dimensions), and lifestyle data (365×8 dimensions), and examples of output include health risk prediction score 0.85, recommended health management plan “improve exercise habits+extend sleep duration,” and anomaly detection flag 1. Based on these outputs, the learning unit automatically extracts health risk factors and lifestyle improvement points for each employee and generates personalized health management plans. Furthermore, during model training, the learning unit maximizes individual optimization accuracy by weighting attribute information and past data, customizing loss functions, and applying transfer learning. Unlike conventional uniform health risk prediction and standardized health management proposals, these processes combine AI-based multidimensional data integration and personalized learning algorithms to achieve highly accurate health risk prediction and countermeasure proposals optimized for each employee's characteristics and risk profile, exhibiting technical effects. Specific application fields include health management for large organizations with diverse ages, genders, and job types, attribute-based risk management for field workers, and individually optimized health monitoring in remote work environments, and can be deployed in various industries and occupations.

The detection unit can estimate the emotions of employees and adjust the criteria for abnormal value detection based on the estimated emotions when detecting changes in employees' health status. For example, when an employee is feeling stressed, the detection criteria for abnormal values are made stricter to enable early detection of anomalies. When an employee is relaxed, the detection criteria for abnormal values are loosened to prevent excessive alerts. Furthermore, when an employee is fatigued, the detection criteria for abnormal values are adjusted to enable detection at appropriate timing. By adjusting the criteria for abnormal value detection based on employees' emotions, more accurate abnormal value detection becomes possible. Specifically, the detection unit inputs facial image data (e.g., 128×128 pixel face images), voice waveform data (e.g., 16 kHz sampled PCM array), and natural language text (e.g., utterances such as “I have been tired recently”), and combines a convolutional neural network (CNN)-based facial expression recognition model, spectrogram conversion+recurrent neural network (RNN)-based voice emotion classification model, and BERT-based natural language processing model to estimate emotion labels (e.g., stress, relaxation, fatigue) and emotion scores (e.g., stress level 0.82, relaxation level 0.15). Based on the estimated emotion scores, the detection unit applies an abnormal value detection criteria control algorithm (e.g., strict abnormal threshold of 0.6 for stress level>0.7, relaxed threshold of 0.8 for relaxation level>0.7), and dynamically adjusts the thresholds for health status change scores and biometric indicator anomaly judgment. Examples of AI input include 10 facial images per day, 5 voice clips, 3 utterance texts, health status change score series (e.g., 0.65, 0.68, 0.82, . . . ), and examples of output include emotion label “stress,” emotion score 0.82, and abnormal value detection threshold 0.6. When the stress level is high, the detection unit makes the criteria for health status change scores, heart rate, sleep duration, etc. stricter, and detects even subtle changes as anomalies. When the relaxation level is high, the threshold is loosened to suppress excessive alert generation. Furthermore, when the fatigue level is high, the threshold is adjusted only for specific indicators (e.g., sleep duration, rest status), enabling fine control according to emotional state. Unlike conventional anomaly detection with fixed thresholds, these processes combine AI-based multimodal emotion estimation and dynamic threshold control algorithms to achieve optimal anomaly detection according to each employee's psychological state and health risk, exhibiting technical effects. Specific application fields include call center operations where stress management is important, safety monitoring for field workers, and health maintenance support in remote work environments, and can be deployed in various industries and occupations.

The guidance unit can estimate the emotions of employees and determine the priority of treatment or consultation destinations to be guided based on the estimated emotions. For example, when an employee is feeling stressed, a stress management expert is prioritized for guidance. When an employee is relaxed, a general health management expert can be guided. Furthermore, when an employee is fatigued, a rest or sleep expert can be prioritized for guidance. By determining the priority of treatment or consultation destinations based on employees' emotions, more appropriate guidance becomes possible. Specifically, the guidance unit inputs facial image data (e.g., 128×128 pixel face images), voice waveform data (e.g., 16 kHz sampled PCM array), and natural language text (e.g., utterances such as “I have been tired recently”), and combines a convolutional neural network (CNN)-based facial expression recognition model, spectrogram conversion+recurrent neural network (RNN)-based voice emotion classification model, and BERT-based natural language processing model to estimate emotion labels (e.g., stress, relaxation, fatigue) and emotion scores (e.g., stress level 0.82, relaxation level 0.15). Based on the estimated emotion labels and scores, the guidance unit applies a guidance priority determination algorithm (e.g., prioritize stress management expert for stress level>0.7, prioritize health management expert for relaxation level>0.7, prioritize rest/sleep expert for fatigue level>0.7), and assigns priorities to the list of treatment proposals or consultation destinations (e.g., occupational physician interview, stress check, recommendation for taking leave, in-house counselor, external consultation desk). Examples of AI input include 10 facial images per day, 5 voice clips, 3 utterance texts, health status vectors (7×10 dimensions), and examples of output include emotion label “stress,” emotion score 0.82, and guidance priority list (e.g., 1st: stress management expert, 2nd: rest recommendation, 3rd: health management expert). Based on these outputs, the guidance unit automatically notifies the employee or supervisor of priority guidance messages (e.g., “Since your stress level is currently high, we will guide you to consult a stress management expert as the highest priority”) via chat tool API or dashboard. Furthermore, the guidance priority is dynamically adjusted in conjunction with each employee's history database according to past consultation history and health status trends. As a technical effect, unlike conventional uniform guidance or subjective judgment, the guidance unit combines AI-based multimodal emotion estimation and priority control algorithms to achieve highly accurate and real-time optimal guidance for treatment or consultation destinations according to the employee's psychological state and health risk. As a result, it contributes to early detection of health risks and prompt response, reduction of employee burden, and operational efficiency. Specific application fields include call center operations where stress management is important, health monitoring for field workers, and mental health support in remote work environments, and can be deployed in various industries and occupations.

The collection unit can estimate the emotions of employees and determine the frequency of data collection based on the estimated emotions of the employees. For example, when an employee is feeling stressed, the frequency of data collection is reduced to alleviate the burden. When an employee is relaxed, the frequency of data collection can be increased to collect more detailed information. Furthermore, when an employee is fatigued, the frequency of data collection can be adjusted to collect data at appropriate timing. By adjusting the frequency of data collection based on employees' emotions, the burden on employees can be reduced. Specifically, the collection unit uses facial image data (e.g., 128×128 pixel RGB tensor face images), voice waveform data (e.g., 1-second 16 kHz sampled PCM array), and transcribed utterance content (e.g., natural language text such as “I have been tired recently”) as input for emotion estimation. The collection unit combines a convolutional neural network (CNN)-based facial expression recognition model, spectrogram conversion+recurrent neural network (RNN)-based voice emotion classification model, and a natural language processing model (e.g., BERT-based emotion classifier) to estimate emotion labels (e.g., stress, relaxation, fatigue) and emotion scores (e.g., stress level 0.78, relaxation level 0.12) from each data type. Examples of AI input include 10 facial images per day, 5 voice clips, and 3 utterance texts, and examples of output include emotion label “stress” and emotion score 0.78. When the estimated emotion score is high (e.g., stress level>0.7), the collection unit dynamically adjusts the collection interval, such as reducing the frequency of data collection from once per day to once every three days. On the other hand, when the relaxation level is high, the collection unit optimizes the frequency according to emotional state, such as collecting detailed data twice per day. These frequency controls are personalized in conjunction with each employee's individual history database, linked to past emotional trends and health status changes. As a technical effect, unlike conventional fixed-interval data collection, the collection unit combines AI-based multimodal emotion estimation and dynamic frequency control algorithms to minimize employees' psychological and physical burden while obtaining highly accurate health data at necessary timing. As a result, data quality and comprehensiveness are improved, and the accuracy of subsequent health status analysis and anomaly detection is greatly enhanced. Specific application fields include call center operations where stress management is important, safety monitoring for field workers, and health maintenance support in remote work environments, and can be deployed in various industries and occupations where both employee psychological burden and data quality are required.

The collection unit can collect not only employees' health status and attendance information, but also data on employees' hobbies and interests. For example, the collection unit collects information on sports and activities that employees enjoy as hobbies, which is useful for understanding health status. The collection unit can also collect information on employees' dietary habits and nutritional intake to support health management. Furthermore, the collection unit can collect data on employees' sleep environment and lifestyle rhythm to enable more accurate understanding of health status. This enables a more comprehensive understanding of employees' health status. Specifically, the collection unit collects health status data (e.g., multidimensional vectors of body temperature, blood pressure, heart rate, sleep duration), attendance information (e.g., clock-in/out times, overtime hours, break status), hobby and interest data (e.g., type of sport, activity frequency, hobby activity time), dietary and nutritional intake data (e.g., food names, quantities, calories, nutrient vectors for three meals per day), sleep environment data (e.g., type of bedding, room temperature, noise level), and lifestyle rhythm data (e.g., wake-up and bedtime, weekday/weekend activity patterns). Examples of AI input include one week of hobby activity records (type of sport and activity time for 7 days), meal records (7×3 meals×10 items), sleep environment data (room temperature and noise level for 7 days), and lifestyle rhythm data (wake-up and bedtime for 7 days). The collection unit integrates these diverse data and utilizes them for multifaceted understanding of health status and early detection of lifestyle risk. For example, the collection unit automatically detects decreases in hobby activity, dietary imbalances, deterioration of sleep environment, and disruption of lifestyle rhythm, and provides highly accurate input data to subsequent learning and detection units. Unlike conventional single-faceted health and attendance data collection, these processes combine AI-based multimodal data integration and lifestyle analysis to enable comprehensive understanding and individual optimization of health risks, exhibiting technical effects. Specific application fields include health support for office workers at high risk of lifestyle-related diseases, safety management for field workers, and lifestyle rhythm optimization in remote work environments, and can be deployed in various industries and occupations.

The learning unit can perform personalized learning based on employees' workplace environment and work details when analyzing collected information. For example, the learning unit detects changes in health status more accurately by considering whether the workplace environment is an office or remote. The learning unit can also propose optimal health management plans based on employees' work details. Furthermore, the learning unit can predict future health risks based on employees' work patterns and workload, enabling employees to take preventive measures against health risks. Specifically, the learning unit integrates and inputs health status data (e.g., multidimensional vectors of body temperature, blood pressure, heart rate, sleep duration), attendance information (e.g., clock-in/out times, overtime hours, break status), workplace environment data (e.g., office, remote, field, business trip), work details data (e.g., type of work, number of meetings attended, workload score), work patterns (e.g., shift work, flextime, fixed work), and workload data (e.g., work volume index, stress score) received from the collection unit. Based on these data, the learning unit combines environment and work detail embedding+multilayer perceptron, time-series prediction models (e.g., LSTM, GRU), and clustering algorithms (e.g., K-means, hierarchical clustering) to perform optimized detection of health status changes and health risk prediction for each employee. Examples of AI input include one year of health status vectors (365×10 dimensions), attendance history (365×4 dimensions), workplace environment labels (365 items), work details data (365 items), work patterns (365 items), and workload scores (365 items), and examples of output include health risk prediction score 0.85, recommended health management plan “reduce workload+recommend remote work,” and anomaly detection flag 1. Based on these outputs, the learning unit automatically extracts health risk factors and work detail improvement points for each employee and generates personalized health management plans. Furthermore, during model training, the learning unit maximizes individual optimization accuracy by weighting workplace environment and work details, customizing loss functions, and applying transfer learning. Unlike conventional uniform health risk prediction and standardized health management proposals, these processes combine AI-based multidimensional data integration and personalized learning algorithms to achieve highly accurate health risk prediction and countermeasure proposals optimized for each employee's workplace environment and work details, exhibiting technical effects. Specific application fields include health management for large organizations with mixed office, field, and remote work, risk management by work details for field workers, and individually optimized health monitoring in remote work environments, and can be deployed in various industries and occupations.

The detection unit can adjust the criteria for abnormal value detection by considering employees' lifestyle habits and family environment when detecting changes in employees' health status. For example, when an employee is feeling stressed due to family circumstances, the criteria for abnormal value detection are made stricter to enable early detection of anomalies. When an employee maintains healthy lifestyle habits, the criteria for abnormal value detection are loosened to prevent excessive alerts. Furthermore, when an employee has an irregular lifestyle rhythm, the criteria for abnormal value detection are adjusted to enable detection at appropriate timing. By adjusting the criteria for abnormal value detection by considering employees' lifestyle habits and family environment, more accurate abnormal value detection becomes possible. Specifically, the detection unit receives as input health status data (e.g., multidimensional vectors of body temperature, blood pressure, heart rate, sleep duration), attendance information (e.g., clock-in/out times, overtime hours, break status), lifestyle habit data (e.g., dietary habits, exercise frequency, sleep score), and family environment data (e.g., family composition, family stress factors, lifestyle rhythm indicators). The detection unit applies lifestyle habit and family environment classification algorithms (e.g., clustering-based lifestyle pattern classification, family environment stress estimation model) and risk factor extraction models (e.g., BERT-based text classifier) to comprehensively evaluate employees' current lifestyle habits and family environment. Examples of AI input include one week of lifestyle habit data (7×5 dimensions), family environment questionnaire (5 items), and health status vectors (7×10 dimensions), and examples of output include “lifestyle habit label: irregular,” “family environment stress level 0.85,” and “abnormal value detection threshold 0.6.” According to lifestyle habits and family environment, the detection unit dynamically adjusts the criteria for abnormal value detection, making the threshold stricter when stress level is high, loosening the threshold when healthy lifestyle habits are maintained, and individually adjusting the threshold for specific indicators in the case of irregular lifestyle rhythm. Unlike conventional uniform abnormal value judgment, these processes apply AI-based lifestyle habit and family environment-linked anomaly detection algorithms to achieve highly accurate anomaly detection optimized for each employee's characteristics and risk profile, exhibiting technical effects. Specific application fields include health management for field workers whose work is affected by family problems, health support for office workers at high risk of lifestyle-related diseases, and lifestyle rhythm optimization in remote work environments, and can be deployed in various industries and occupations.

The guidance unit can estimate the emotions of employees and determine the display method for treatments or consultation destinations to be guided based on the estimated emotions. For example, if an employee is feeling stressed, a simple and highly visible display method is provided. If the employee is relaxed, a display method including detailed information can also be provided. Furthermore, if the employee is in a hurry, a display method emphasizing key points can be provided. By adjusting the display method based on the employee's emotions, highly visible guidance can be achieved. Specifically, the guidance unit receives as input facial image data (e.g., 128×128 pixel face images), voice waveform data (e.g., 16 kHz sampling), and natural language text (e.g., utterances such as “I am in a hurry”), and combines a CNN-based facial recognition model, an RNN-based voice emotion classification model, and a BERT-based natural language processing model to estimate emotion labels (e.g., stress, relaxation, fatigue, tension) and emotion scores (e.g., stress level 0.78, relaxation level 0.12, tension level 0.65). The guidance unit applies a guidance display control algorithm (e.g., simple display for stress level>0.7, detailed display for relaxation level>0.7, key point emphasis display for tension level>0.6) based on the estimated emotion scores, and dynamically adjusts the display layout, information volume, and highlight colors of dashboards and notification screens. Examples of AI input include 10 face images per day, 5 voice clips, 3 utterance texts, and a guidance content list (e.g., guidance to stress management experts, health management guidance), while output examples include display format “simple”, highlighted item “priority guidance destination”, and notification message “Caution: High stress level”. When the stress level is high, the guidance unit provides a simple display that prominently emphasizes only the guidance destination, minimizing employee burden and confusion. On the other hand, when the relaxation level is high, a rich display including detailed breakdowns of guidance destinations, past trend graphs, and countermeasures is provided. Furthermore, when the tension or urgency level is high, only the key points are prominently displayed to support rapid decision-making. These display controls, unlike conventional fixed layouts or uniform information displays, combine AI-based emotion estimation and dynamic UI optimization algorithms to achieve optimal information presentation tailored to each employee's psychological state and work situation, thereby providing a technical effect. Specific application fields include health monitoring for field workers where stress management is important, mental health support for office workers, and health maintenance support in remote work environments, and can be deployed in various industries and occupations.

The collection unit can preferentially collect highly relevant data based on the geographic location information of employees, in addition to health status and attendance information. For example, when an employee is in the office, data related to working hours and work details are collected. When the employee is at home, data related to health status and rest can also be collected. Furthermore, when the employee is on a business trip, data related to travel can also be collected. By preferentially collecting highly relevant data considering the geographic location information of employees, more accurate information can be obtained. Specifically, the collection unit acquires GPS coordinate data (e.g., latitude and longitude pairs), Wi-Fi/Bluetooth beacon information, and location labels from location information service APIs (e.g., office, home, business trip destination) in real time from employees' smartphones or wearable devices. The collection unit applies location classification algorithms (e.g., clustering for stay location determination, time-series location estimation) using this location information as input to accurately identify the current location of employees. Examples of AI input include a series of GPS coordinates for the past 24 hours (e.g., 1440×2 dimensions), a list of Wi-Fi beacon IDs, and location label “office”; output examples include “Current location: office”, “Relevant data collection: working hours, work details”, and “Priority: high”. When the current location is the office, the collection unit preferentially collects work-related data such as working hours, work details, and meeting participation status; when at home, it collects data such as sleep duration, rest status, and home environment; and when on a business trip, it preferentially acquires data such as travel distance, means of transportation, and health status at the business trip destination. This location-linked data collection is personalized in conjunction with each employee's work history and health status history. As a technical effect, unlike conventional uniform data collection, the collection unit combines AI-based location information analysis and relevant data priority control to efficiently acquire optimal data according to the employee's activity location and work situation. As a result, the comprehensiveness and reliability of the data are improved, and changes in health status and risk signs can be accurately grasped. Specific application fields include safety management for field workers, health monitoring in remote work environments, and health risk management for business trips, and can be deployed in various industries and occupations.

Below, the processing flow of Example of the Embodiment is briefly described. Specifically, the present system operates in cooperation among the collection unit, learning unit, detection unit, and guidance unit modules. First, the collection unit collects multidimensional data such as employees' health status data (e.g., body temperature, blood pressure, heart rate, sleep duration, stress score), attendance information (e.g., clock-in/clock-out times, overtime hours, break status), lifestyle data (e.g., meal content, exercise frequency, sleep score), emotion estimation data (e.g., face images, voice waveforms, utterance texts), geographic location information (e.g., GPS coordinates, location labels), and social media activity data (e.g., post texts, images, interaction information). Next, the learning unit performs personalized learning considering attribute information, workplace environment, work details, lifestyle habits, and emotional state, using the multidimensional data received from the collection unit as input, and constructs models for detecting changes in health status and predicting health risks. The learning unit combines time-series prediction models (e.g., LSTM), clustering, attribute embedding plus multilayer perceptron, and natural language processing models to extract risk factors and health management plans for each employee. The detection unit uses the models constructed by the learning unit to compare the latest collected data with past data, applies abnormal value detection algorithms (e.g., autoencoders, anomaly scoring, dynamic threshold control) and threshold adjustment based on emotion estimation results, and detects changes in health status and risk signs with high accuracy. Furthermore, the detection unit realizes complex anomaly detection by combining various factors such as lifestyle habits, home environment, geographic location, and social media activity. The guidance unit automatically generates optimal treatment proposals and consultation destination lists based on changes in health status, abnormal factors, emotion estimation results, past consultation history, and geographic location information detected by the detection unit, and notifies personalized guidance messages to the employee and their supervisor via chat tool APIs or dashboards. The guidance unit dynamically adjusts the display method and guidance priority according to emotional state and urgency, supporting prompt and accurate health risk response. This series of processing flows, unlike conventional simple health and attendance management systems, combines AI-based multidimensional data integration, personalized learning, dynamic anomaly detection, and real-time guidance optimization to simultaneously achieve early detection of health risks, rapid response, reduction of employee burden, and improved work efficiency, thereby providing a technical effect. Specific application fields include safety management for field workers in manufacturing, health support for remote workers in IT companies, and multi-professional collaborative health monitoring in medical institutions, and can be deployed in various industries, occupations, and operational forms.

Step 1: The collection unit collects health status or attendance information of employees. Health status of employees includes, for example, body temperature, blood pressure, heart rate, but is not limited to these examples. Attendance information includes, for example, clock-in time, clock-out time, break time, but is not limited to these examples. The collection unit collects, for example, health status and attendance information entered daily by employees. Step 2: The learning unit learns the information collected by the collection unit. The learning unit analyzes the collected information and learns patterns using, for example, machine learning or deep learning. Step 3: The detection unit detects changes in health status based on the information learned by the learning unit. The detection unit uses, for example, an algorithm that detects abnormal values by comparing with past data. Step 4: The guidance unit provides guidance on appropriate treatment or consultation destinations based on changes in health status detected by the detection unit. The guidance unit proposes appropriate treatment based on, for example, pre-learned expert advice or information on counseling services. Specifically, in Step 1, the system collects multidimensional data such as health status data (e.g., body temperature, blood pressure, heart rate, sleep duration, stress score), attendance information (e.g., clock-in/clock-out times, overtime hours, break status), lifestyle data (e.g., meal content, exercise frequency, sleep score), emotion estimation data (e.g., face images, voice waveforms, utterance texts), geographic location information (e.g., GPS coordinates, location labels), and social media activity data (e.g., post texts, images, interaction information). In Step 2, the learning unit performs personalized learning considering attribute information, workplace environment, work details, lifestyle habits, and emotional state, using these multidimensional data as input, and constructs models for detecting changes in health status and predicting health risks. The learning unit combines time-series prediction models (e.g., LSTM), clustering, attribute embedding plus multilayer perceptron, and natural language processing models to extract risk factors and health management plans for each employee. In Step 3, the detection unit uses the models constructed by the learning unit to compare the latest collected data with past data, applies abnormal value detection algorithms (e.g., autoencoders, anomaly scoring, dynamic threshold control) and threshold adjustment based on emotion estimation results, and detects changes in health status and risk signs with high accuracy. Furthermore, the detection unit realizes complex anomaly detection by combining various factors such as lifestyle habits, home environment, geographic location, and social media activity. In Step 4, the guidance unit automatically generates optimal treatment proposals and consultation destination lists based on changes in health status, abnormal factors, emotion estimation results, past consultation history, and geographic location information detected by the detection unit, and notifies personalized guidance messages to the employee and their supervisor via chat tool APIs or dashboards. The guidance unit dynamically adjusts the display method and guidance priority according to emotional state and urgency, supporting prompt and accurate health risk response. This series of processing flows, unlike conventional simple health and attendance management systems, combines AI-based multidimensional data integration, personalized learning, dynamic anomaly detection, and real-time guidance optimization to simultaneously achieve early detection of health risks, rapid response, reduction of employee burden, and improved work efficiency, thereby providing a technical effect. Specific application fields include safety management for field workers in manufacturing, health support for remote workers in IT companies, and multi-professional collaborative health monitoring in medical institutions, and can be deployed in various industries, occupations, and operational forms.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the above-described elements, including the collection unit, learning unit, detection unit, and guidance unit, is implemented by at least one of, for example, the smart device 14 and the data processing apparatus 12. For example, the collection unit is implemented by a control unit 46A of the smart device 14 and collects health status and attendance information input daily by employees. The learning unit is implemented, for example, by a specific processing unit 290 of the data processing apparatus 12 and analyzes the collected information using machine learning or deep learning to learn patterns. The detection unit is implemented, for example, by the specific processing unit 290 of the data processing apparatus 12 and detects changes in health status based on the learned information. The guidance unit is implemented, for example, by the specific processing unit 290 of the data processing apparatus 12 and provides guidance on appropriate treatment or consultation destinations based on the detected changes in health status. The correspondence between each unit and the apparatus or control unit is not limited to the above examples and various modifications are possible.

### Second Embodiment

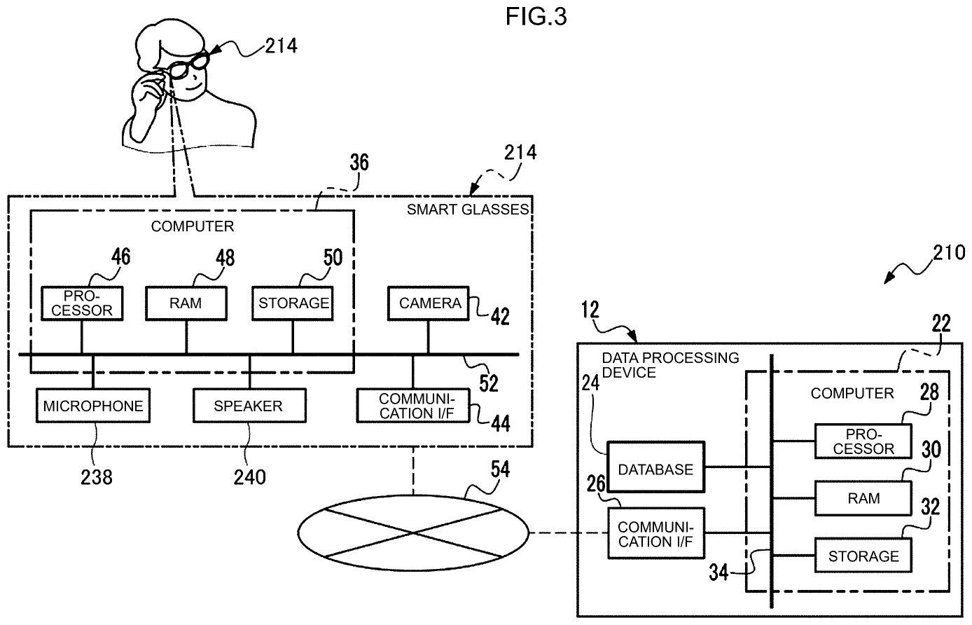

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

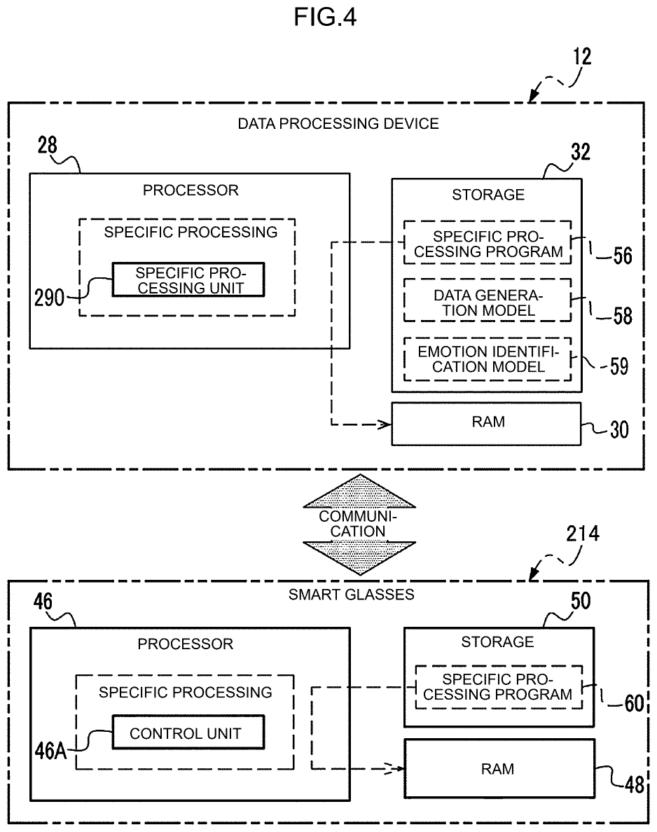

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the above-described elements, including the collection unit, learning unit, detection unit, and guidance unit, is implemented by at least one of, for example, the smart glasses 214 and the data processing apparatus 12. For example, the collection unit is implemented by a control unit 46A of the smart glasses 214 and collects health status and attendance information input daily by employees. The learning unit is implemented, for example, by a specific processing unit 290 of the data processing apparatus 12 and analyzes the collected information using machine learning or deep learning to learn patterns. The detection unit is implemented, for example, by the specific processing unit 290 of the data processing apparatus 12 and detects changes in health status based on the learned information. The guidance unit is implemented, for example, by the specific processing unit 290 of the data processing apparatus 12 and provides guidance on appropriate treatment or consultation destinations based on the detected changes in health status. The correspondence between each unit and the apparatus or control unit is not limited to the above examples and various modifications are possible.

### Third Embodiment

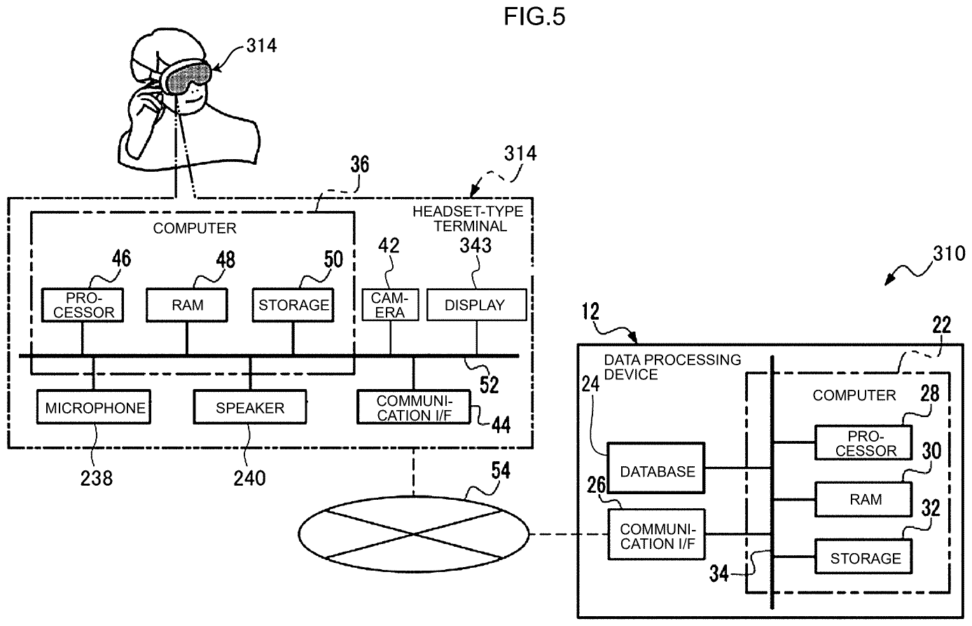

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

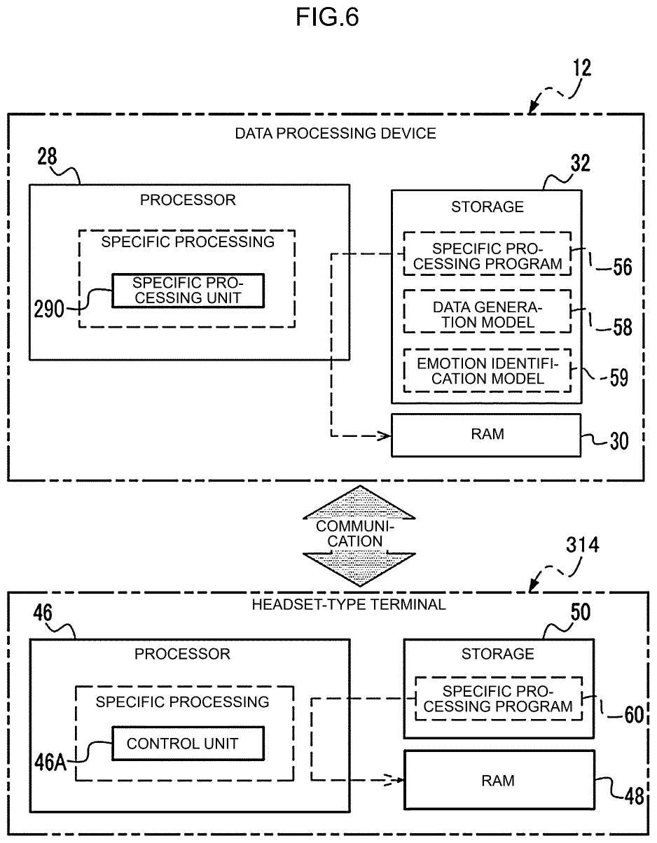

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the above-described elements, including the collection unit, learning unit, detection unit, and guidance unit, is implemented by at least one of, for example, the headset-type terminal 314 and the data processing apparatus 12. For example, the collection unit is implemented by a control unit 46A of the headset-type terminal 314 and collects health status and attendance information input daily by employees. The learning unit is implemented, for example, by a specific processing unit 290 of the data processing apparatus 12 and analyzes the collected information using machine learning or deep learning to learn patterns. The detection unit is implemented, for example, by the specific processing unit 290 of the data processing apparatus 12 and detects changes in health status based on the learned information. The guidance unit is implemented, for example, by the specific processing unit 290 of the data processing apparatus 12 and provides guidance on appropriate treatment or consultation destinations based on the detected changes in health status. The correspondence between each unit and the apparatus or control unit is not limited to the above examples and various modifications are possible.

### Fourth Embodiment

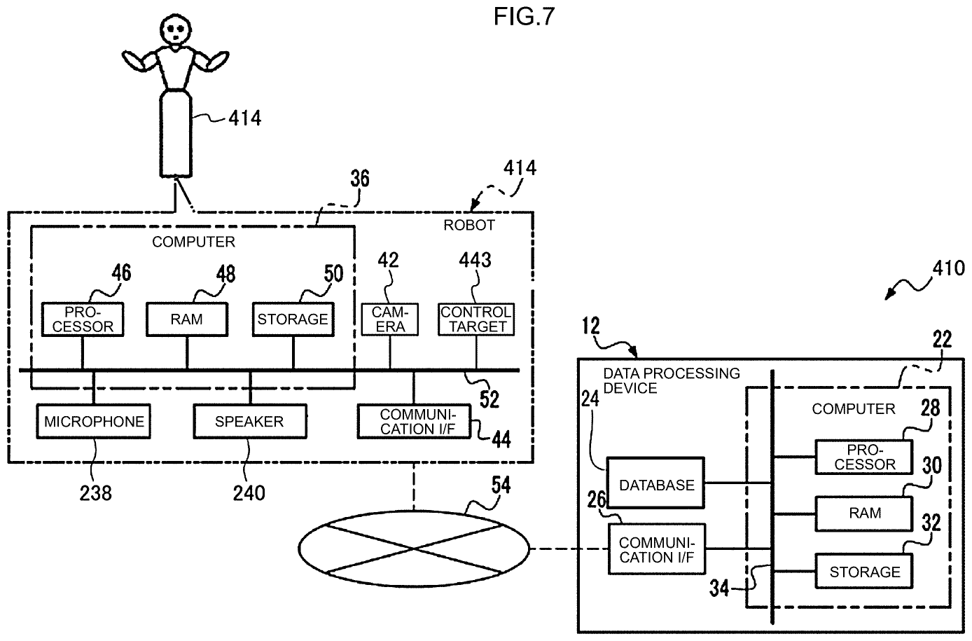

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

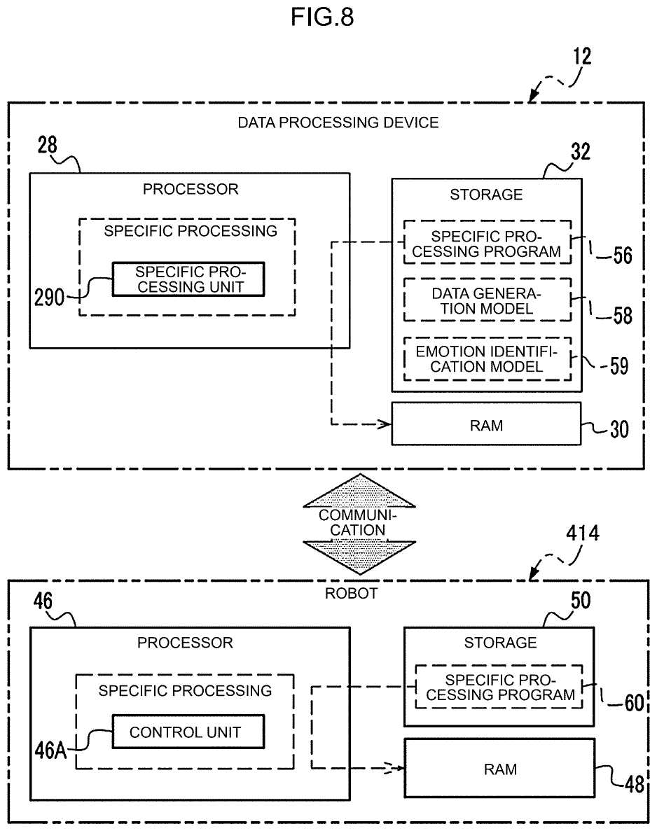

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the above-described elements, including the collection unit, learning unit, detection unit, and guidance unit, is implemented by at least one of, for example, the robot 414 and the data processing apparatus 12. For example, the collection unit is implemented by a control unit 46A of the robot 414 and collects health status and attendance information input daily by employees. The learning unit is implemented, for example, by a specific processing unit 290 of the data processing apparatus 12 and analyzes the collected information using machine learning or deep learning to learn patterns. The detection unit is implemented, for example, by the specific processing unit 290 of the data processing apparatus 12 and detects changes in health status based on the learned information. The guidance unit is implemented, for example, by the specific processing unit 290 of the data processing apparatus 12 and provides guidance on appropriate treatment or consultation destinations based on the detected changes in health status. The correspondence between each unit and the apparatus or control unit is not limited to the above examples and various modifications are possible.

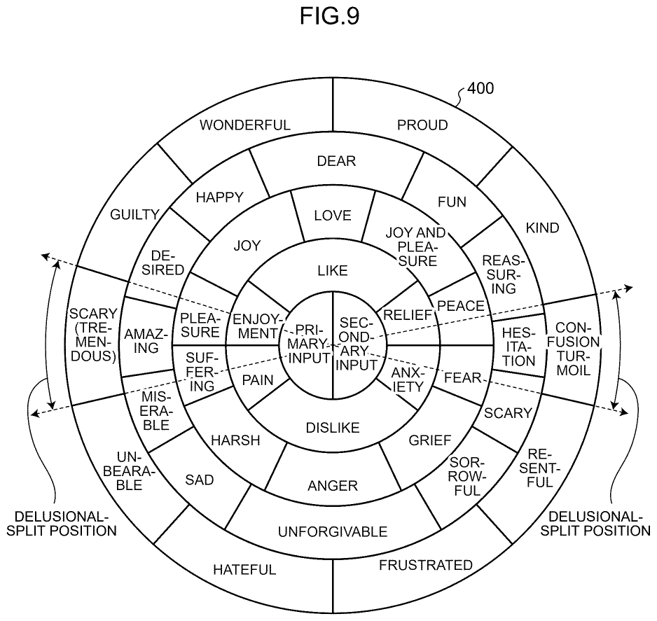

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

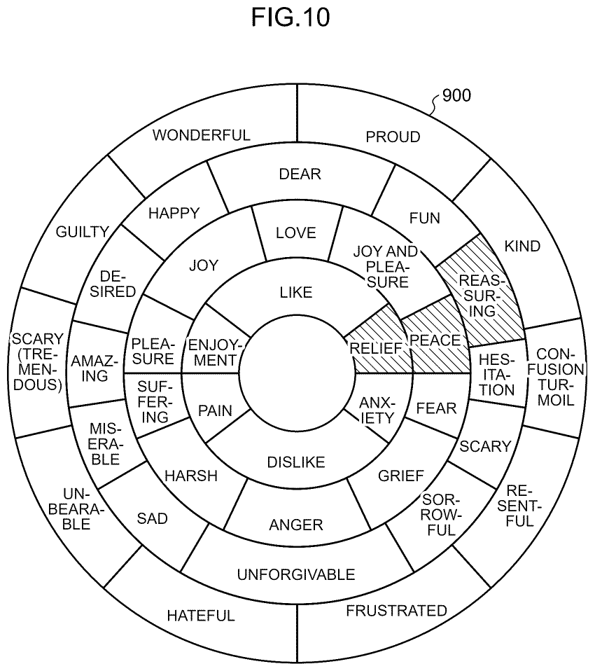

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1)A system comprising: a collection unit configured to collect health status or attendance information of employees; a learning unit configured to learn information collected by the collection unit; a detection unit configured to detect changes in health status based on information learned by the learning unit; and a guidance unit configured to provide guidance on treatment or consultation destinations based on changes in health status detected by the detection unit.
(Supplementary Note 2)The system according to Supplementary Note 1, wherein the collection unit is configured to collect detailed data on employees' sleep duration, physical condition, working hours, and work details.
(Supplementary Note 3)The system according to Supplementary Note 1, wherein the learning unit is configured to analyze the collected information and learn patterns.
(Supplementary Note 4)The system according to Supplementary Note 1, wherein the detection unit uses an algorithm to detect abnormal values by comparing with past data.
(Supplementary Note 5)The system according to Supplementary Note 1, wherein the guidance unit proposes appropriate treatment based on pre-learned expert advice or information on counseling services.
(Supplementary Note 6)The system according to Supplementary Note 1, wherein the collection unit estimates the emotions of employees and determines the frequency of data collection based on the estimated emotions of the employees.
(Supplementary Note 7)The system according to Supplementary Note 1, wherein the collection unit analyzes employees' past health status or attendance information and selects an appropriate data collection method.
(Supplementary Note 8)The system according to Supplementary Note 1, wherein the collection unit performs filtering during data collection based on the employees' current projects or workload.
(Supplementary Note 9)The system according to Supplementary Note 1, wherein the collection unit estimates the emotions of employees and determines the priority of data to be collected based on the estimated emotions of the employees.
(Supplementary Note 10)The system according to Supplementary Note 1, wherein the collection unit preferentially collects highly relevant data during data collection based on the geographic location information of employees.
(Supplementary Note 11)The system according to Supplementary Note 1, wherein the collection unit analyzes employees' social media activities during data collection and collects relevant data.
(Supplementary Note 12)The system according to Supplementary Note 1, wherein the learning unit estimates the emotions of employees and selects learning data based on the estimated emotions of the employees.
(Supplementary Note 13)The system according to Supplementary Note 1, wherein the learning unit determines the level of detail of learning during learning based on the importance of the collected data.
(Supplementary Note 14)The system according to Supplementary Note 1, wherein the learning unit uses different learning algorithms during learning according to the category of the data.
(Supplementary Note 15)The system according to Supplementary Note 1, wherein the learning unit estimates the emotions of employees and determines the frequency of learning based on the estimated emotions of the employees.
(Supplementary Note 16)The system according to Supplementary Note 1, wherein the learning unit weights learning data during learning based on the timing of data collection.
(Supplementary Note 17)The system according to Supplementary Note 1, wherein the learning unit determines the order of learning during learning based on the relevance of the data.
(Supplementary Note 18)The system according to Supplementary Note 1, wherein the detection unit estimates the emotions of employees and determines the criteria for detecting abnormal values based on the estimated emotions of the employees.
(Supplementary Note 19)The system according to Supplementary Note 1, wherein the detection unit improves the accuracy of abnormal value detection during detection based on the interrelationships of the data.
(Supplementary Note 20)The system according to Supplementary Note 1, wherein the detection unit detects abnormal values during detection based on employees' attribute information.
(Supplementary Note 21)The system according to Supplementary Note 1, wherein the detection unit estimates the emotions of employees and determines the display method of abnormal values based on the estimated emotions of the employees.
(Supplementary Note 22)The system according to Supplementary Note 1, wherein the detection unit detects abnormal values during detection based on the geographic distribution of the data.
(Supplementary Note 23)The system according to Supplementary Note 1, wherein the detection unit improves the accuracy of abnormal value detection during detection based on related literature.
(Supplementary Note 24)The system according to Supplementary Note 1, wherein the guidance unit estimates the emotions of employees and determines the priority of treatment or consultation destinations to be guided based on the estimated emotions of the employees.
(Supplementary Note 25)The system according to Supplementary Note 1, wherein the guidance unit analyzes employees' past health status or attendance information during guidance and selects appropriate treatment or consultation destinations.
(Supplementary Note 26)The system according to Supplementary Note 1, wherein the guidance unit customizes treatment or consultation destinations during guidance based on employees' current living conditions.
(Supplementary Note 27)The system according to Supplementary Note 1, wherein the guidance unit estimates the emotions of employees and determines the display method of treatment or consultation destinations to be guided based on the estimated emotions of the employees.
(Supplementary Note 28)The system according to Supplementary Note 1, wherein the guidance unit selects appropriate treatment or consultation destinations during guidance based on the geographic location information of employees.
(Supplementary Note 29)The system according to Supplementary Note 1, wherein the guidance unit analyzes employees' social media activities during guidance and proposes treatment or consultation destinations.

What is claimed is:

1. A system comprising:
a communication interface configured to communicate with a client terminal via a packet-switched network;
a memory storing a data generation model obtained by deep learning on a neural network, and an emotion identification model;
a database; and
circuitry configured to:
receive, from the client terminal via the communication interface, structured data comprising at least one of biometric data, timestamp data, or activity data, and store the structured data in the database;
analyze the structured data stored in the database using a time-series prediction model comprising at least one of a recurrent neural network or a long short-term memory network to extract a pattern feature vector;
detect an anomaly by comparing the pattern feature vector with reference data stored in the database and generating an anomaly score indicating a degree of deviation;
estimate an emotion of a user by applying the emotion identification model to sensor data received from the client terminal via the communication interface;
generate, using the data generation model, inference data comprising at least one of a recommendation text or an action proposal, based on the detected anomaly and the estimated emotion; and transmit the inference data to the client terminal via the communication interface and the packet-switched network, the inference data causing the client terminal to present the inference data to the user.

2. The system according to claim 1, wherein the structured data comprises health status data of a user, the health status data comprising at least one of body temperature, blood pressure, heart rate, or sleep duration.

3. The system according to claim 1, wherein the structured data further comprises attendance data comprising at least one of clock-in time, clock-out time, overtime hours, or break status.

4. The system according to claim 1, wherein the circuitry is further configured to preprocess the structured data by performing at least one of missing value imputation, outlier removal, or normalization before storing the structured data in the database.

5. The system according to claim 1, wherein the time-series prediction model comprises a Transformer model with a self-attention mechanism, and wherein the circuitry is configured to extract the pattern feature vector by applying feature extraction layers to multidimensional time-series tensor data.

6. The system according to claim 1, wherein the circuitry is further configured to detect the anomaly by applying at least one of threshold judgment, autoencoder-based anomaly scoring, or rule-based branching to the anomaly score.

7. The system according to claim 1, wherein the circuitry is further configured to adjust a frequency of receiving the structured data from the client terminal based on the estimated emotion, such that when the estimated emotion indicates stress, the circuitry reduces the frequency, and when the estimated emotion indicates relaxation, the circuitry increases the frequency.

8. The system according to claim 1, wherein the circuitry is further configured to analyze past structured data stored in the database associated with the user to select an optimal data collection method using at least one of a decision tree, a random forest, or a reinforcement learning model.

9. The system according to claim 1, wherein the circuitry is further configured to determine a priority of the structured data to be received based on the estimated emotion, such that when the estimated emotion indicates stress, the circuitry prioritizes structured data having a high importance attribute.

10. The system according to claim 1, wherein the circuitry is further configured to receive geographic location information from the client terminal via the communication interface, and to preferentially receive structured data associated with a geographic region corresponding to the geographic location information.

11. The system according to claim 1, wherein the circuitry is further configured to adjust a threshold for the anomaly score based on the estimated emotion, such that when the estimated emotion indicates stress, the circuitry applies a stricter threshold, and when the estimated emotion indicates relaxation, the circuitry applies a relaxed threshold.

12. The system according to claim 1, wherein the circuitry is further configured to detect the anomaly based on interrelationships among the structured data by applying at least one of correlation analysis, co-occurrence network analysis, or a multivariate anomaly detection model.

13. The system according to claim 1, wherein the circuitry is further configured to detect the anomaly based on attribute information of the user comprising at least one of age, gender, or occupation by referring to an attribute-specific normal range database.

14. The system according to claim 1, wherein the circuitry is further configured to adjust a display method of the inference data based on the estimated emotion, such that when the estimated emotion indicates stress, the circuitry generates the inference data in a simplified format, and when the estimated emotion indicates relaxation, the circuitry generates the inference data in a detailed format.

15. The system according to claim 1, wherein the circuitry is further configured to determine a priority of the inference data based on the estimated emotion, such that when the estimated emotion indicates stress, the circuitry prioritizes inference data associated with a high urgency attribute.

16. The system according to claim 1, wherein the circuitry is further configured to analyze past inference data stored in the database associated with the user, and to select a format of the inference data based on the past inference data by analyzing content trends and user preferences.

17. The system according to claim 1, wherein the circuitry is further configured to select the inference data based on geographic location information of the user received from the client terminal, such that when the user is at a first location, the circuitry generates inference data associated with nearby resources, and when the user is at a second location, the circuitry generates inference data associated with remote resources.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a camera having a CMOS image sensor, and a display;
a processor;
a random-access memory;
a memory storing a data generation model obtained by deep learning on a neural network, and an emotion identification model;
a database; and
circuitry configured to:
receive, from the client terminal via the communication interface, structured data comprising at least one of biometric data, timestamp data, or activity data, and store the structured data in the database;
analyze the structured data stored in the database using a time-series prediction model comprising at least one of a recurrent neural network, a long short-term memory network, or a Transformer model to extract a pattern feature vector;
detect an anomaly by comparing the pattern feature vector with reference data stored in the database and generating an anomaly score;
estimate an emotion of the user by applying the emotion identification model to at least one of voice data captured by the microphone or image data captured by the camera;
generate, using the data generation model, inference data comprising at least one of a recommendation text or an action proposal, based on the detected anomaly and the estimated emotion;
adjust at least one of a format, a level of detail, or a priority of the inference data based on the estimated emotion; and
transmit the inference data to the client terminal via the communication interface, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a data processing system comprising a communication interface, a memory storing a data generation model obtained by deep learning on a neural network and an emotion identification model, and a database, the method comprising:
receiving, from a client terminal via the communication interface and a packet-switched network, structured data comprising at least one of biometric data, timestamp data, or activity data, and storing the structured data in the database;
analyzing the structured data stored in the database using a time-series prediction model comprising at least one of a recurrent neural network or a long short-term memory network to extract a pattern feature vector;
detecting an anomaly by comparing the pattern feature vector with reference data stored in the database and generating an anomaly score indicating a degree of deviation;
estimating an emotion of a user by applying the emotion identification model to sensor data received from the client terminal via the communication interface;
generating, using the data generation model, inference data comprising at least one of a recommendation text or an action proposal, based on the detected anomaly and the estimated emotion; and
transmitting the inference data to the client terminal via the communication interface and the packet-switched network, the inference data causing the client terminal to present the inference data to the user.
